SEQUENCE LISTING (93 sequences)

SEQ ID NO: 1 - DNA, 9865 bp, synthetic construct
  misc_feature 1..9865: note = Synthetic Nucleotide
  source 1..9865: mol_type = other DNA
aacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcctaccgttcgtatagcatacattatacgaacggtacttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgctcacgacgagttgtaatctagggcggccgccagcttgggcccgaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtccgacgcggcccgacgggtccgaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatcagatctaacatccataatcgtattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggactaaagaaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaagatcttccacaaggaattcgtggtcattattctgcccaagatgattacaatttgtgtttatcggttcaacctttcattgaatctgtagatgagacaacaggccaagaatttttcaaacctctgaaaggtgtatttgatgacttggaatctcgctttcctcaccatacaaagacttcctggagagacagattcagaaagtttgcctctaaatacggtgttcgtcagtacatcgcgtattatgaaaagactgttgaactcaatggtgttcctaatccgatgacgaactttacctcaaaggcttccattgaaaaatttagagaaagacgcgggacttcacgtaacagtggccttccaggcccggttggtgtagaagctgtaagctctttggaccacatatccccattggtcacatctaattccaattctgcagctgctgcagctgctgccgcagcagttgcagcctctgcctctgcttcttcagctcctaatacttcaactaccaatttctttgaacaggagaatattgcccaagttctctctgcacataacaacgagcagtctattgcagaagttattgagtccgcacagaatgtcaacacccatgaaagtgaacctatagctgatcatgttcgaaaaaatcttacagacgatgaattgcttgacaaaatggatgatattttaagctccagaagtctaggcggactagatgacttgataaagatcctctacactgagctgggatttgctcatcgttataccgaatttctttttacctcatgttctggtgatgtgattttcttccgaccattagtggaacatttccttcttactggtgagtgggagctggagaatactcgtggcatctggaccggtcgtcaagacgaaatgctacgtgctagcaatctagatgacctgcacaagttaattgacctgcatgggaaagaacgtgttgagaccagaagaaaagccatcaagggagaatgatcataagaaatgaaaaacgtataagt

SEQ ID NO: 2 - protein, 404 aa, synthetic construct
  REGION 1..404: note = Pichia pastoris
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 3 - protein, 388 aa, synthetic construct
  REGION 1..388: note = Pichia pastoris
MRSDLTSIIVFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 4 - protein, 5 aa, synthetic construct
  REGION 1..5: note = Synthetic Peptide
AKADG

SEQ ID NO: 5 - protein, 6 aa, synthetic construct
  REGION 1..6: note = Synthetic Peptide
AKADGS

SEQ ID NO: 6 - protein, 7 aa, synthetic construct
  REGION 1..7: note = Synthetic Peptide
AKADGSL

SEQ ID NO: 7 - protein, 8 aa, synthetic construct
  REGION 1..8: note = Synthetic Peptide
AKADGSLL

SEQ ID NO: 8 - protein, 9 aa, synthetic construct
  REGION 1..9: note = Synthetic Peptide
AKADGSLLY

SEQ ID NO: 9 - protein, 10 aa, synthetic construct
  REGION 1..10: note = Synthetic Peptide
AKADGSLLYY

SEQ ID NO: 10 - protein, 6 aa, synthetic construct
  REGION 1..6: note = Synthetic Peptide
MAKADG

SEQ ID NO: 11 - protein, 7 aa, synthetic construct
  REGION 1..7: note = Synthetic Peptide
MAKADGS

SEQ ID NO: 12 - protein, 8 aa, synthetic construct
  REGION 1..8: note = Synthetic Peptide
MAKADGSL

SEQ ID NO: 13 - protein, 9 aa, synthetic construct
  REGION 1..9: note = Synthetic Peptide
MAKADGSLL

SEQ ID NO: 14 - protein, 10 aa, synthetic construct
  REGION 1..10: note = Synthetic Peptide
MAKADGSLLY

SEQ ID NO: 15 - protein, 11 aa, synthetic construct
  REGION 1..11: note = Synthetic Peptide
MAKADGSLLYY

SEQ ID NO: 16 - protein, 12 aa, synthetic construct
  REGION 1..12: note = Synthetic Peptide
MAKADGSLLYYN

SEQ ID NO: 17 - protein, 13 aa, synthetic construct
  REGION 1..13: note = Synthetic Peptide
MAKADGSLLYYNP

SEQ ID NO: 18 - protein, 14 aa, synthetic construct
  REGION 1..14: note = Synthetic Peptide
MAKADGSLLYYNPH

SEQ ID NO: 19 - protein, 15 aa, synthetic construct
  REGION 1..15: note = Synthetic Peptide
MAKADGSLLYYNPHN

SEQ ID NO: 20 - protein, 16 aa, synthetic construct
  REGION 1..16: note = Synthetic Peptide
MAKADGSLLYYNPHNP

SEQ ID NO: 21 - protein, 17 aa, synthetic construct
  REGION 1..17: note = Synthetic Peptide
MAKADGSLLYYNPHNPP

SEQ ID NO: 22 - protein, 18 aa, synthetic construct
  REGION 1..18: note = Synthetic Peptide
MAKADGSLLYYNPHNPPR

SEQ ID NO: 23 - protein, 19 aa, synthetic construct
  REGION 1..19: note = Synthetic Peptide
MAKADGSLLYYNPHNPPRR

SEQ ID NO: 24 - protein, 20 aa, synthetic construct
  REGION 1..20: note = Synthetic Peptide
MAKADGSLLYYNPHNPPRRY

SEQ ID NO: 25 - protein, 21 aa, synthetic construct
  REGION 1..21: note = Synthetic Peptide
MAKADGSLLYYNPHNPPRRYY

SEQ ID NO: 26 - protein, 24 aa, synthetic construct
  REGION 1..24: note = Synthetic Peptide
AKADGSLLYYNPHNPPRRYYFYMA

SEQ ID NO: 27 - protein, 25 aa, synthetic construct
  REGION 1..25: note = Synthetic Peptide
MAKADGSLLYYNPHNPPRRYYFYMA

SEQ ID NO: 28 - protein, 9 aa, synthetic construct
  REGION 1..9: note = Synthetic Peptide
RSDLTSIIV

SEQ ID NO: 29 - DNA, 15 bp, synthetic construct
  misc_feature 1..15: note = Synthetic Oligonucleotide
  source 1..15: mol_type = other DNA
gcgaaggcagatggc

SEQ ID NO: 30 - DNA, 18 bp, synthetic construct
  misc_feature 1..18: note = synthetic oligonucleotide
  source 1..18: mol_type = other DNA
gcgaaggcagatggcagt

SEQ ID NO: 31 - DNA, 21 bp, synthetic construct
  misc_feature 1..21: note = Synthetic Oligonucleotide
  source 1..21: mol_type = other DNA
gcgaaggcagatggcagtttg

SEQ ID NO: 32 - DNA, 24 bp, synthetic construct
  misc_feature 1..24: note = Synthetic Oligonucleotide
  source 1..24: mol_type = other DNA
gcgaaggcagatggcagtttgctc

SEQ ID NO: 33 - DNA, 27 bp, synthetic construct
  misc_feature 1..27: note = Synthetic Oligonucleotide
  source 1..27: mol_type = other DNA
gcgaaggcagatggcagtttgctctac

SEQ ID NO: 34 - DNA, 30 bp, synthetic construct
  misc_feature 1..30: note = Synthetic Oligonucleotide
  source 1..30: mol_type = other DNA
gcgaaggcagatggcagtttgctctactat

SEQ ID NO: 35 - DNA, 18 bp, synthetic construct
  misc_feature 1..18: note = Synthetic Oligonucleotide
  source 1..18: mol_type = other DNA
atggcgaaggcagatggc

SEQ ID NO: 36 - DNA, 21 bp, synthetic construct
  misc_feature 1..21: note = Synthetic Oligonucleotide
  source 1..21: mol_type = other DNA
atggcgaaggcagatggcagt

SEQ ID NO: 37 - DNA, 24 bp, synthetic construct
  misc_feature 1..24: note = Synthetic Oligonucleotide
  source 1..24: mol_type = other DNA
atggcgaaggcagatggcagtttg

SEQ ID NO: 38 - DNA, 27 bp, synthetic construct
  misc_feature 1..27: note = Synthetic Oligonucleotide
  source 1..27: mol_type = other DNA
atggcgaaggcagatggcagtttgctc

SEQ ID NO: 39 - DNA, 30 bp, synthetic construct
  misc_feature 1..30: note = Synthetic Oligonucleotide
  source 1..30: mol_type = other DNA
atggcgaaggcagatggcagtttgctctac

SEQ ID NO: 40 - DNA, 30 bp, synthetic construct; identical to SEQ ID NO: 39
  misc_feature 1..30: note = Synthetic Oligonucleotide'
  source 1..30: mol_type = other DNA

SEQ ID NO: 41 - DNA, 36 bp, synthetic construct
  misc_feature 1..36: note = Synthetic Oligonucleotide
  source 1..36: mol_type = other DNA
atggcgaaggcagatggcagtttgctctactataat

SEQ ID NO: 42 - DNA, 39 bp, synthetic construct
  misc_feature 1..39: note = Synthetic Oligonucleotide
  source 1..39: mol_type = other DNA
atggcgaaggcagatggcagtttgctctactataatcct

SEQ ID NO: 43 - DNA, 42 bp, synthetic construct
  misc_feature 1..42: note = Synthetic Oligonucleotide
  source 1..42: mol_type = other DNA
atggcgaaggcagatggcagtttgctctactataatcctcac

SEQ ID NO: 44 - DNA, 42 bp, synthetic construct; identical to SEQ ID NO: 43
  misc_feature 1..42: note = Synthetic Oligonucleotide
  source 1..42: mol_type = other DNA

SEQ ID NO: 45 - DNA, 48 bp, synthetic construct
  misc_feature 1..48: note = Synthetic Oligonucleotide
  source 1..48: mol_type = other DNA
atggcgaaggcagatggcagtttgctctactataatcctcacaatcca

SEQ ID NO: 46 - DNA, 51 bp, synthetic construct
  misc_feature 1..51: note = Synthetic Oligonucleotide
  source 1..51: mol_type = other DNA
atggcgaaggcagatggcagtttgctctactataatcctcacaatccaccc

SEQ ID NO: 47 - DNA, 54 bp, synthetic construct
  misc_feature 1..54: note = Synthetic Oligonucleotide
  source 1..54: mol_type = other DNA
atggcgaaggcagatggcagtttgctctactataatcctcacaatccacccaga

SEQ ID NO: 48 - DNA, 57 bp, synthetic construct
  misc_feature 1..57: note = Synthetic Oligonucleotide
  source 1..57: mol_type = other DNA
atggcgaaggcagatggcagtttgctctactataatcctcacaatccacccagaagg

SEQ ID NO: 49 - DNA, 60 bp, synthetic construct
  misc_feature 1..60: note = Synthetic Oligonucleotide
  source 1..60: mol_type = other DNA
atggcgaaggcagatggcagtttgctctactataatcctcacaatccacccagaaggtat

SEQ ID NO: 50 - DNA, 63 bp, synthetic construct
  misc_feature 1..63: note = Synthetic Oligonucleotide
  source 1..63: mol_type = other DNA
atggcgaaggcagatggcagtttgctctactataatcctcacaatccacccagaaggtattac

SEQ ID NO: 51 - DNA, 73 bp, synthetic construct
  misc_feature 1..73: note = Synthetic Oligonucleotide
  source 1..73: mol_type = other DNA
gcgaaggcagatggcagtttgctctactataatcctcacaatccacccagaaggtattacttctacatggcta

SEQ ID NO: 52 - DNA, 76 bp, synthetic construct
  misc_feature 1..76: note = Synthetic Oligonucleotide
  source 1..76: mol_type = other DNA
atggcgaaggcagatggcagtttgctctactataatcctcacaatccacccagaaggtattacttctacatggcta

SEQ ID NO: 53 - DNA, 10742 bp, synthetic construct
  misc_feature 1..10742: note = Synthetic Oligonucleotide
  source 1..10742: mol_type = other DNA
aacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctctagatgctcaccgcaatgctgttaaggttcgtatggagaaactgggacttatttaattatttagagattttaacttacatttagattcgatagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctgatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggatcctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttggtgccgactcgaccattgggcatctcggctctcacccgtcgacgcgcaaggacttgacctttttgtcttcgtacaacggacagtctacgtcgccaaactcaggacatttggccagttttggcggtggcaacttcatcttgggaggcattctcctgaacgagcaaaagtacattgactttggaatcaagcttgccagctcgtactttggcacgtacacccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttgagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaccggcgggaacaaatttgtctttaacacggaggcgcacccctttagcatccgttcatcatcacgacggggcggccaccttgctcacgacgagttgtaatctagggcggccgccagcttgggcccgaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtccgacgcggcccgacgggtccgaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatcagatctaacatccataatcgtattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggactaaagaaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaagatcttccacaaggaattcgtggtcattattctgcccaagatgattacaatttgtgtttatcggttcaacctttcattgaatctgtagatgagacaacaggccaagaatttttcaaacctctgaaaggtgtatttgatgacttggaatctcgctttcctcaccatacaaagacttcctggagagacagattcagaaagtttgcctctaaatacggtgttcgtcagtacatcgcgtattatgaaaagactgttgaactcaatggtgttcctaatccgatgacgaactttacctcaaaggcttccattgaaaaatttagagaaagacgcgggacttcacgtaacagtggccttccaggcccggttggtgtagaagctgtaagctctttggaccacatatccccattggtcacatctaattccaattctgcagctgctgcagctgctgccgcagcagttgcagcctctgcctctgcttcttcagctcctaatacttcaactaccaatttctttgaacaggagaatattgcccaagttctctctgcacataacaacgagcagtctattgcagaagttattgagtccgcacagaatgtcaacacccatgaaagtgaacctatagctgatcatgttcgaaaaaatcttacagacgatgaattgcttgacaaaatggatgatattttaagctccagaagtctaggcggactagatgacttgataaagatcctctacactgagctgggatttgctcatcgttataccgaatttctttttacctcatgttctggtgatgtgattttcttccgaccattagtggaacatttccttcttactggtgagtgggagctggagaatactcgtggcatctggaccggtcgtcaagacgaaatgctacgtgctagcaatctagatgacctgcacaagttaattgacctgcatgggaaagaacgtgttgagaccagaagaaaagccatcaagggagaatgatcataagaaatgaaaaacgtataagt

SEQ ID NO: 54 - protein, 173 aa, synthetic construct
  REGION 1..173: note = Synthetic Peptide
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEALDAHRNAVKVRMEKLGLI

SEQ ID NO: 55 - DNA, 522 bp, synthetic construct
  misc_feature 1..522: note = Synthetic Oligonucleotide
  source 1..522: mol_type = other DNA
atggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctctagatgctcaccgcaatgctgttaaggttcgtatggagaaactgggacttatttaa

SEQ ID NO: 56 - protein, 388 aa, synthetic construct; identical to SEQ ID NO: 3
  REGION 1..388: note = Synthetic Peptide

SEQ ID NO: 57 - DNA, 1167 bp, synthetic construct
  misc_feature 1..1167: note = Synthetic Oligonucleotide
  source 1..1167: mol_type = other DNA
atgagatcagatctaacatccataatcgtattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggactaa

SEQ ID NO: 58 - DNA, 4311 bp, synthetic construct
  misc_feature 1..4311: note = Synthetic Oligonucleotide
  source 1..4311: mol_type = other DNA
ttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcctaccgttcgtatagcatacattatacgaagttatggatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattgggtaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgacacgttcgtttgtgcaagcttcaacgatgccaaaagggtataataagcgtcatttgcagcattgtgaagaaaactatgtggcaagccaagcctgcgaagaatgtagtcgagaattgagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgaccactttggatgatactgcttacagatacagaacttctgttccaggtgatgctgaagctattgaagctttggatggatctttcaccactgatactgttttcagagtcactgctactggtgatggattcactttgagagaagttcctgttgatcctcctttgaccaaagtttttcctgatgatgaatctgatgatgaatctgatgctggtgaagatggtgatccagattctagaacttttgttgcttatggtgatgatggtgatttggctggatttgttgttgtttcttattctggatggaacagaagattgactgttgaagatattgaagttgctccagaacatagaggtcatggtgttggaagagctttgatgggattggcaactgagtttgccagagaaagaggtgctggtcatctttggttggaagtcaccaatgtcaatgctccagctattcatgcttacagaagaatgggattcactctttgtggattggatactgctttgtatgatggaactgcttctgatggagaacaagctttgtacatgtccatgccatgtccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcataacttcgtatagcatacattatacgaacggtacttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgccacaaaacgtggatctcccaaccctacgagggcgg

SEQ ID NO: 59 - DNA, 12722 bp, synthetic construct
  misc_feature 1..12722: note = Synthetic Oligonucleotide
  source 1..12722: mol_type = other DNA
aacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcctaccgttcgtatagcatacattatacgaagttatggatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattgggtaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgacacgttcgtttgtgcaagcttcaacgatgccaaaagggtataataagcgtcatttgcagcattgtgaagaaaactatgtggcaagccaagcctgcgaagaatgtagtcgagaattgagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgaccactttggatgatactgcttacagatacagaacttctgttccaggtgatgctgaagctattgaagctttggatggatctttcaccactgatactgttttcagagtcactgctactggtgatggattcactttgagagaagttcctgttgatcctcctttgaccaaagtttttcctgatgatgaatctgatgatgaatctgatgctggtgaagatggtgatccagattctagaacttttgttgcttatggtgatgatggtgatttggctggatttgttgttgtttcttattctggatggaacagaagattgactgttgaagatattgaagttgctccagaacatagaggtcatggtgttggaagagctttgatgggattggcaactgagtttgccagagaaagaggtgctggtcatctttggttggaagtcaccaatgtcaatgctccagctattcatgcttacagaagaatgggattcactctttgtggattggatactgctttgtatgatggaactgcttctgatggagaacaagctttgtacatgtccatgccatgtccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcataacttcgtatagcatacattatacgaacggtacttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggatcctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttggtgccgactcgaccattgggcatctcggctctcacccgtcgacgcgcaaggacttgacctttttgtcttcgtacaacggacagtctacgtcgccaaactcaggacatttggccagttttggcggtggcaacttcatcttgggaggcattctcctgaacgagcaaaagtacattgactttggaatcaagcttgccagctcgtactttggcacgtacacccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttgagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaccggcgggaacaaatttgtctttaacacggaggcgcacccctttagcatccgttcatcatcacgacggggcggccaccttgctcacgacgagttgtaatctagggcggccgccagcttgggcccgaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtccgacgcggcccgacgggtccgaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatcagatctaacatccataatcgtattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggactaaagaaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaagatcttccacaaggaattcgtggtcattattctgcccaagatgattacaatttgtgtttatcggttcaacctttcattgaatctgtagatgagacaacaggccaagaatttttcaaacctctgaaaggtgtatttgatgacttggaatctcgctttcctcaccatacaaagacttcctggagagacagattcagaaagtttgcctctaaatacggtgttcgtcagtacatcgcgtattatgaaaagactgttgaactcaatggtgttcctaatccgatgacgaactttacctcaaaggcttccattgaaaaatttagagaaagacgcgggacttcacgtaacagtggccttccaggcccggttggtgtagaagctgtaagctctttggaccacatatccccattggtcacatctaattccaattctgcagctgctgcagctgctgccgcagcagttgcagcctctgcctctgcttcttcagctcctaatacttcaactaccaatttctttgaacaggagaatattgcccaagttctctctgcacataacaacgagcagtctattgcagaagttattgagtccgcacagaatgtcaacacccatgaaagtgaacctatagctgatcatgttcgaaaaaatcttacagacgatgaattgcttgacaaaatggatgatattttaagctccagaagtctaggcggactagatgacttgataaagatcctctacactgagctgggatttgctcatcgttataccgaatttctttttacctcatgttctggtgatgtgattttcttccgaccattagtggaacatttccttcttactggtgagtgggagctggagaatactcgtggcatctggaccggtcgtcaagacgaaatgctacgtgctagcaatctagatgacctgcacaagttaattgacctgcatgggaaagaacgtgttgagaccagaagaaaagccatcaagggagaatgatcataagaaatgaaaaacgtataagt

SEQ ID NO: 60 - DNA, 19 bp, synthetic construct
  misc_feature 1..19: note = Synthetic Oligonucleotide
  source 1..19: mol_type = other DNA
caagttgcgccccctggca

SEQ ID NO: 61 - DNA, 21 bp, synthetic construct
  misc_feature 1..21: note = Synthetic Oligonucleotide
  source 1..21: mol_type = other DNA
tggagcagctaatgcggagga

SEQ ID NO: 62 - DNA, 21 bp, synthetic construct
  misc_feature 1..21: note = Synthetic Oligonucleotide
  source 1..21: mol_type = other DNA
agttccgccgagacttcccca

SEQ ID NO: 63 - DNA, 21 bp, synthetic construct
  misc_feature 1..21: note = Synthetic Oligonucleotide
  source 1..21: mol_type = other DNA
ttcagccggaatttgtgccgt

SEQ ID NO: 64 - DNA, 20 bp, synthetic construct
  misc_feature 1..20: note = Synthetic Oligonucleotide
  source 1..20: mol_type = other DNA
atccagggtgacggtgccga

SEQ ID NO: 65 - DNA, 20 bp, synthetic construct
  misc_feature 1..20: note = Synthetic Oligonucleotide
  source 1..20: mol_type = other DNA
gcaagaggcccggcagtacc

SEQ ID NO: 66 - DNA, 21 bp, synthetic construct
  misc_feature 1..21: note = Synthetic Oligonucleotide
  source 1..21: mol_type = other DNA
ccgccctcgtagggttgggag

SEQ ID NO: 67 - DNA, 19 bp, synthetic construct
  misc_feature 1..19: note = Synthetic Oligonucleotide
  source 1..19: mol_type = other DNA
ttcgcggtcgggtcacaca

SEQ ID NO: 68 - DNA, 20 bp, synthetic construct
  misc_feature 1..20: note = Synthetic Oligonucleotide
  source 1..20: mol_type = other DNA
aactgccatctgccttcgcc

SEQ ID NO: 69 - DNA, 20 bp, synthetic construct
  misc_feature 1..20: note = Synthetic Oligonucleotide
  source 1..20: mol_type = other DNA
caaatcgcgggttcgcggtc

SEQ ID NO: 70 - DNA, 23 bp, synthetic construct
  misc_feature 1..23: note = Synthetic Oligonucleotide
  source 1..23: mol_type = other DNA
gagcaaactgccatctgccttcg

SEQ ID NO: 71 - DNA, 75 bp, synthetic construct
  misc_feature 1..75: note = Synthetic Oligonucelotide
  source 1..75: mol_type = other DNA
gtgttcgtagcaaatatcatcagcctaccgttcgtatagcatacattatacgaagttatggatctaacatccaaa

SEQ ID NO: 72 - DNA, 75 bp, synthetic construct
  misc_feature 1..75: note = Synthetic Oligonucelotide
  source 1..75: mol_type = other DNA
tttggatgttagatccataacttcgtataatgtatgctatacgaacggtaggctgatgatatttgctacgaacac

SEQ ID NO: 73 - DNA, 75 bp, synthetic construct
  misc_feature 1..75: note = Synthetic Oligonucelotide
  source 1..75: mol_type = other DNA
gccgccatccagtgtcataacttcgtatagcatacattatacgaacggtacttttttgtagaaatgtcttggtgt

SEQ ID NO: 74 - DNA, 75 bp, synthetic construct
  misc_feature 1..75: note = Synthetic Oligonucleotide
  source 1..75: mol_type = other DNA
acaccaagacatttctacaaaaaagtaccgttcgtataatgtatgctatacgaagttatgacactggatggcggc

SEQ ID NO: 75 - DNA, 30 bp, synthetic construct
  misc_feature 1..30: note = Synthetic Oligonucleotide
  source 1..30: mol_type = other DNA
gtgttcgtagcaaatatcatcagcctaccg

SEQ ID NO: 76 - DNA, 31 bp, synthetic construct
  misc_feature 1..31: note = Synthetic Oligonucleotide
  source 1..31: mol_type = other DNA
acaccaagacatttctacaaaaaagtaccgt

SEQ ID NO: 77 - DNA, 20 bp, synthetic construct
  misc_feature 1..20: note = Synthetic Oligonucleotide
  source 1..20: mol_type = other DNA
ttcgcggtcgggtcacacac

SEQ ID NO: 78 - DNA, 23 bp, synthetic construct
  misc_feature 1..23: note = Synthetic Oligonucleotide
  source 1..23: mol_type = other DNA
ggagcagctaatgcggaggatgc

SEQ ID NO: 79 - DNA, 20 bp, synthetic construct
  misc_feature 1..20: note = Synthetic Oligonucleotide
  source 1..20: mol_type = other DNA
cggtcgggtcacacacggag

SEQ ID NO: 80 - DNA, 21 bp, synthetic construct
  misc_feature 1..21: note = Synthetic Oligonucleotide
  source 1..21: mol_type = other DNA
tggagcagctaatgcggagga

SEQ ID NO: 81 - DNA, 21 bp, synthetic construct
  misc_feature 1..21: note = Synthetic Oligonucleotide
  source 1..21: mol_type = other DNA
tgagtcctggtgctcctgacg

SEQ ID NO: 82 - DNA, 20 bp, synthetic construct
  misc_feature 1..20: note = Synthetic Oligonucleotide
  source 1..20: mol_type = other DNA
cccctcctgttgcgtttggc

SEQ ID NO: 83 - DNA, 21 bp, synthetic construct
  misc_feature 1..21: note = Synthetic Oligonucleotide
  source 1..21: mol_type = other DNA
agcgttctgagtcctggtgct

SEQ ID NO: 84 - DNA, 20 bp, synthetic construct
  misc_feature 1..20: note = Synthetic Oligonucleotide
  source 1..20: mol_type = other DNA
ggtcctgcgtttgcaacggt

SEQ ID NO: 85 - DNA, 22 bp, synthetic construct
  misc_feature 1..22: note = Synthetic Oligonucleotide
  source 1..22: mol_type = other DNA
actaacgccgccatccagtgtc

SEQ ID NO: 86 - DNA, 22 bp, synthetic construct
  misc_feature 1..22: note = Synthetic Oligonucleotide
  source 1..22: mol_type = other DNA
gcttcagccggaatttgtgccg

SEQ ID NO: 87 - DNA, 20 bp, synthetic construct
  misc_feature 1..20: note = Synthetic Oligonucleotide
  source 1..20: mol_type = other DNA
cgcctcgacatcatctgccc

SEQ ID NO: 88 - DNA, 20 bp, synthetic construct
  misc_feature 1..20: note = Synthetic Oligonucleotide
  source 1..20: mol_type = other DNA
tcagccggaatttgtgccgt

SEQ ID NO: 89 - protein, 23 aa, synthetic construct
  REGION 1..23: note = Synthetic Peptide
FYMAIFAVSVICVLYGPSQQLSS

SEQ ID NO: 90 - protein, 5 aa, synthetic construct
  REGION 1..5: note = Synthetic Peptide
FYMAI

SEQ ID NO: 91 - protein, 4 aa, synthetic construct
  REGION 1..4: note = Synthetic Peptide
HDEL

SEQ ID NO: 92 - protein, 14 aa, synthetic construct
  REGION 1..14: note = Synthetic Peptide
LFARGGLYADMDTM

SEQ ID NO: 93 - protein, 14 aa, synthetic construct
  REGION 1..14: note = Synthetic Peptide
GKPIPNPLLGLDST

(19) United States
(12) Patent Application Publication
(10) Pub. No.: US 2025/0304980 A1
(43) Pub. Date: Oct. 2, 2025

(54) PICHIA PASTORIS STRAINS FOR PRODUCING PREDOMINANTLY HOMOGENEOUS GLYCAN STRUCTURE

(71) Applicant: Research Corporation Technologies, Inc., Tucson, AZ (US)

(72) Inventors: Kurt R. Gehlsen, Tucson, AZ (US); Thomas G. Chappell, San Marcos, CA (US)

(73) Assignee: Research Corporation Technologies, Inc., Tucson, AZ (US)

(21) Appl. No.: 19/232,956

(22) Filed: Jun. 10, 2025

Related U.S. Application Data
(63) Continuation of application No. 18/520,960, filed on Nov. 28, 2023, now Pat. No. 12,359,211.
Application No. 18/520,960 is a continuation of application No. 17/528,619, filed on Nov. 17, 2021, now Pat. No. 11,866,715.
Application No. 17/528,619 is a continuation of application No. 16/801,466, filed on Feb. 26, 2020, now Pat. No. 11,220,692.
Application No. 16/801,466 is a continuation of application No. 16/404,838, filed on May 7, 2019, now Pat. No. 10,612,033.
Application No. 16/404,838 is a continuation of application No. 15/444,870, filed on Feb. 28, 2017, now Pat. No. 10,329,572.
Application No. 15/444,870 is a continuation of application No. 14/437,683, filed on Apr. 22, 2015, now Pat. No. 9,617,550.
(60) Provisional application No. 61/717,423, filed on Oct. 23, 2012.

(51) Int. Cl. C12N 15/81; C12N 9/10; C12N 9/24

(52) U.S. Cl. CPC C12N 15/815; C12N 9/1051; C12N 9/2488; C12Y 204/01232; C12Y 302/0113

(57) ABSTRACT

Disclosed herein are novel Pichia pastoris strains for expression of exogenous proteins with substantially homogeneous N-glycans. The strains are genetically engineered to include a mutant OCH1 allele which is transcribed into an mRNA coding for a mutant OCH1 gene product (i.e., α-1,6-mannosyltransferase, or “OCH1 protein”). The mutant OCH1protein contains a catalytic domain substantially identical to that of the wild type OCH1 protein, but lacks an N-terminal sequence necessary to target the OCH1 protein to the Golgi apparatus. The strains disclosed herein are robust, stable, and transformable, and the mutant OCH1 allele and the ability to produce substantially homogeneous N-glycans are maintained for generations after rounds of freezing and thawing and after subsequent transformations.

### CROSS REFERENCE TO RELATED APPLICATIONS

This application is a continuation of U.S. patent application Ser. No. 18/520,960, filed Nov. 28, 2023, which is a continuation of U.S. patent application Ser. No. 17/528,619, filed Nov. 17, 2021, now U.S. Pat. No. 11,866,715, which is a continuation of U.S. patent application Ser. No. 16/801,466, filed Feb. 26, 2020, now U.S. Pat. No. 11,220,692, which is a continuation of U.S. patent application Ser. No. 16/404,838, filed May 7, 2019, now U.S. Pat. No. 10,612,033, which is a continuation of U.S. patent application Ser. No. 15/444,870, filed Feb. 28, 2017, now U.S. Pat. No. 10,329,572, which is a continuation of U.S. patent application Ser. No. 14/437,683, filed Apr. 22, 2015, now U.S. Pat. No. 9,617,550, which is a 371 of International application having Serial No. PCT/US2013/066335, filed on Oct. 23, 2013, which claims the benefit of priority from U.S. Provisional Application No. 61/717,423, filed Oct. 23, 2012, the entire contents of which are incorporated herein by reference.

### INCORPORATION BY REFERENCE OF SEQUENCE LISTING

The sequence listing in the XML format, named as 30272ZYXWVU_SequenceListing.xml of 159,744 bytes, created on Jun. 4, 2025, and submitted to the United States Patent and Trademark Office via Patent Center, is incorporated herein by reference.

### BACKGROUND OF THE INVENTION

Pichia pastoris is a highly successful system for production of a wide variety of recombinant proteins. Several factors have contributed to its rapid acceptance, including: (1) a promoter derived from the alcohol oxidase I (AOX1) gene of P. pastoris that is uniquely suited for the controlled expression of foreign genes; (2) the similarity of techniques needed for the molecular genetic manipulation of P. pastoris to those of Saccharomyces cerevisiae; and (3) the strong preference of P. pastoris for respiratory growth, a physiological trait that facilitates its culturing at high-cell densities relative to fermentative yeasts.

As a yeast, P. pastoris is a single-celled microorganism that is easy to manipulate and culture. However, it is also a eukaryote and capable of many of the post-translational modifications performed by higher eukaryotic cells such as proteolytic processing, folding, disulfide bond formation and glycosylation. Thus, many proteins that would end up as inactive inclusion bodies in bacterial systems are produced as biologically active molecules in P. pastoris. The P. pastoris system is also generally regarded as being faster, easier, and less expensive to use than expression systems derived from higher eukaryotes such as insect and mammalian tissue culture cell systems and usually gives higher expression levels.

P. pastoris has the potential of performing many of the posttranslational modifications typically associated with higher eukaryotes. These include processing of signal sequences (both pre- and prepro-type), folding, disulfide bridge formation, and both O- and N-linked glycosylation. Glycosylation of secreted foreign (higher) eukaryotic proteins by P. pastoris and other fungi can be problematic. In mammals, O-linked oligosaccharides are composed of a variety of sugars including N-acetylgalactosamine, galactose and sialic acid. In contrast, lower eukaryotes, including P. pastoris, may add O-oligosaccharides solely composed of mannose (Man) residues.

N-glycosylation in P. pastoris is also different than in higher eukaryotes. In all eukaryotes, it begins in the ER with the transfer of a lipid-linked oligosaccharide unit, Glc3Man9GlcNAc2 (Glc=glucose; GlcNAc=N-acetylglucosamine), to asparagine at the recognition sequence Asn-X-Ser/Thr. This oligosaccharide core unit is subsequently trimmed to Man8GlcNAc2. It is at this point that lower and higher eukaryotic glycosylation patterns begin to differ. The mammalian Golgi apparatus performs a series of trimming and addition reactions that generate oligosaccharides composed of either Man5-6GlcNAc2 (high-mannose type), a mixture of several different sugars (complex type) or a combination of both (hybrid type). Two distinct patterns of N-glycosylation have been observed on foreign proteins secreted by P. pastoris. Some proteins are secreted with carbohydrate structures similar in size and structure to the core unit (Man8-11GlcNAc2). Other foreign proteins secreted from P. pastoris receive much more carbohydrate and appear to be hyperglycosylated.

N-linked high mannose oligosaccharides added to proteins by yeasts represent a problem in the use of foreign secreted proteins by the pharmaceutical industry. For example, they can be exceedingly antigenic when introduced intravenously into mammals and furthermore may cause rapid clearance of the protein from the blood by the liver.

In an attempt to modify the N-glycosylation pathway of Pichia pastoris, a strain (hereinafter referred to as “M5-Blast”) was created, as described in Jacobs et al., 2009, Nature Protocols 4:58-70. M5-Blast is a modification of the P. pastoris GS115 strain wherein the endogenous mannosyltransferase gene OCH1 is disrupted by the introduction of a cassette comprising an α-1,2 mannosidase gene. However, the M5-Blast strain is subject to genomic rearrangements that regenerate the endogenous OCH1 gene and in parallel remove the α-1,2 mannosidase gene after rounds of freezing and thawing, growth under various temperatures and conditions, and from subsequent transformations with other plasmids to introduce exogenous genes.

### SUMMARY OF THE DISCLOSURE

Disclosed herein are novel Pichia pastoris strains for expression of exogenous proteins with substantially homogeneous N-glycans. More specifically, the strains are genetically engineered to include a mutant OCH1 allele which is transcribed into an mRNA coding for a mutant OCH1 gene product (i.e., α-1,6-mannosyltransferase, or “OCH1 protein”). The mutant OCH1protein contains a catalytic domain substantially identical to that of the wild type OCH1 protein, but has an N-terminal sequence that alters the localization of the OCH1 protein to or in the Golgi apparatus. The strains do not include any other OCH1 allele that produces an mRNA coding for a functional OCH1 protein. Such strains are robust, stable, and transformable, and the mutant OCH1 allele and the associated phenotype (i.e., ability to produce substantially homogeneous N-glycans) are maintained for generations, after rounds of freezing and thawing, and after subsequent transformations.

This disclosure also features methods of constructing the strains, as well as methods of expressing proteins via the strains.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1. Diagram of deletion strategy for removing the OCH1 N-terminal fragment homology from M5-Blast Pichia genome. Homology arms flanking the OCH1 N-terminal fragment are used to create a double crossover construct containing lox71-lox66 recombination sites. The intervening sequences can be removed by cre mediated recombination.

FIG. 2. PCR primers for amplification of flanking arms of double crossover construct from M5-Blast genomic DNA.

FIG. 3. PCR reactions for the addition of lox sites to the ends of the homology arms.

FIG. 4. Addition of M5-Blast genomic DNA extensions onto existing lox71-MazF-Nat^{R}-lox66 cassette.

FIG. 5. Overlap assembly and amplification of the final sequence for generating the double crossover fragment for M5-Blast Pichia transformation. This final construct has >500 bp of homology arms flanking the selection/counter-selection cassette.

FIG. 6. PCR primer pairs used to generate DNA fragment for double crossover recombination event.

FIG. 7. Theoretical arrangement of LEU5-mannosidaseHDEL region of the M5-Blast genome after double crossover recombination event.

FIG. 8. Theoretical arrangement of genomic DNA between LEU5 and mannosidaseHDEL after cre recombination.

FIG. 9. PCR primers 81487118-80765854 used to verify DNA sequence of region that could have been derived from PCR products transformed into the M5-Blast strain.

FIG. 10. N-glycan analysis of a recombinant protein expressed in various P. pastoris strains.

FIG. 11. N-glycan analysis of trastuzumab obtained in Study 1 described in Example 6.

FIG. 12. Comparison of Her2 binding affinity of Man5-type trastuzumab (Study 1) and commercial Herceptin by ELISA.

FIG. 13. DSA-FACE analysis of the total N-glycan pool on medium proteins from ‘Trans’ strains. A. result for a malto-dextrose reference. Panel B to F show results for N-glycans, as follows: B. GS Trans strain in microscale; C. M5 Trans strain in microscale; D. GS Trans strain in bioreactor; E. M5 Trans strain in bioreactor; F. reference N-glycans from bovine RNase B.

FIG. 14. DSA-FACE analysis of the total N-glycan pool on medium proteins from ‘CalB’ strains. A. result for a malto-dextrose reference. Panel B to F show results for N-glycans, as follows: B. GS CalB strain in microscale; C. M5 CalB strain in microscale; D. GS CalB strain in bioreactor; E. M5 CalB strain in bioreactor; F. reference N-glycans from bovine RNase B.

FIG. 15. DSA-FACE analysis of the total N-glycan pool on medium proteins from ‘CalA’ strains. A. result for a malto-dextrose reference. Panel B to F show results for N-glycans, as follows: B. GS CalA strain in microscale; C. M5 CalA strain in microscale; D. GS CalA strain in bioreactor; E. M5 CalA strain in bioreactor; F. reference N-glycans from bovine RNase B.

Table 1 lists the DNA sequence (SEQ ID NO: 1) of the OCH1 locus in a SuperM5 strain described in Example 1.

Table 2 lists the amino acid sequence for wild type OCH1 (SEQ ID NO: 2) in Pichia pastoris.

Table 3 lists nucleotides that may be deleted from the Upstream OCH1 segment.

Table 4 lists the DNA sequence for the OCH1 locus (+/−2 kb) for the M5-Blast Pichia pastoris strain.

Table 5 lists the amino acid sequence and nucleotide sequence for the Upstream OCH1 segment.

Table 6 lists the amino acid sequence and nucleotide sequence for the Downstream OCH1 segment.

Table 7. N-glycan analysis of trastuzumab obtained from Study 2 (Example 6).

Table 8. Kinetic parameters of trastuzumab analyzed on BIAcore (Example 6).

### DETAILED DESCRIPTION

Genetically Engineered Pichia pastoris Strains

This disclosure features novel genetically engineered Pichia pastoris strains which are robust, stable, and transformable, and which produce proteins with substantially homogeneous N-glycan structures.

As further described herein, the strains are genetically engineered to include a mutant OCH1 allele which is transcribed into an mRNA coding for a mutant OCH1 gene product (i.e., α-1,6-mannosyltransferase, or “OCH1 protein”). The mutant OCH1protein contains a catalytic domain substantially identical to that of the wild type OCH1 protein, but has an N-terminal sequence that alters the localization of the OCH1 protein to or in the Golgi apparatus. The strains do not include any other OCH1 allele that produces an mRNA coding for a functional OCH1 protein.

The strains can be additionally genetically engineered to contain a nucleic acid coding for and expressing an α-1,2-mannosidase which converts the M8 N-glycan, Man8GlcNAc2, to the M5 N-glycan, Man5GlcNAc2.

As a result of the genetic modifications, the strains disclosed herein produce substantially homogeneous N-glycans.

By “substantially homogeneous” N-glycans it is meant that given a preparation containing a population of a particular glycoprotein of interest, at least 50%, 60%, 75%, 80%, 85%, 90% or even 95% of the N-glycans on the protein molecules within the population are the same.

By “predominant N-glycan structure” or “predominant glycoform” it is meant a specific N-glycan structure or glycoform of (i.e., attached to) a protein constitutes the greatest percentage of all N-glycan structures or glycoforms of the protein. In certain specific embodiments, a predominant glycoform accounts for at least 40%, 50%, 60%, 70%, 80%, 90% or 95% or greater of the population of all glycoforms on the protein. Examples of desirable N-glycan structures include, e.g., Man8GlcNAc2 (or “M8”) or Man5GlcNAc2(“M5”). Additional desirable N-glycan structures include, GnM5 (GlcNAcMan₅GlcNAc₂), GalGnM5 (GalGlcNAcMan₅GlcNAc₂), GalGnM3 (GalGlcNAcMan₃GlcNAc₂), GnM3 (GlcNAcMan₃GlcNAc₂), Gn2M3 (GlcNAc₂Man₃GlcNAc₂), and Gal2Gn2M3 (Gal₂GlcNAc₂Man₃GlcNAc₂). The structures of these N-glycans have been described, e.g., in Jacobs et al., 2009, Nature Protocols 4:58-70, incorporated herein by reference.

In a specific embodiment, the strains of this invention include both a mutant OCH1 allele and a nucleic acid coding for and expressing an α-1,2-mannosidase, such that the strains produce homogeneous N-glycans with M5 being the predominant glycoform. These strains are also referred to herein as SuperM5 or SuperMan5 strains. An example of a SuperM5 strain is described in the Example section below.

The strains of this invention are “robust”, which means that the strains (unless noted otherwise as an auxotroph or deficient strain, e.g., protease deficient, AOX1 mutant, etc.) have approximately the same growth rate and the same growth conditions as unmodified Pichia pastoris strains such as strain GS115. For example, the strains of this invention can grow at elevated temperatures (e.g., 30° C., 37° C. or even 42° C.) and are not temperature sensitive. For example, the SuperM5 strains disclosed herein are robust and are not temperature sensitive.

The strains of this invention are also stable, which means that the genetic modifications and the phenotype as a result of the genetic modifications (i.e., producing homogeneous N-glycans) are maintained through generations, e.g., at least 10, 20, 30, 40 or 50 generations (cell divisions), after rounds of freezing and thawing, and after subsequent transformations. For example, the SuperM5 strains disclosed herein maintain the mutant OCH1 allele through generations and are able to continue making substantially homogeneous M8 (or other downstream N-glycans), without reversion.

### Genetic Engineering—Mutant OCH1 Allele

The strains of this invention are genetically engineered to include a mutant OCH1 allele which is transcribed into an mRNA coding for a mutant OCH1 gene product (i.e., α-1,6-mannosyltransferase, or the “OCH1 protein”). The mutant OCH1 protein contains a catalytic domain substantially identical to that of the wild type OCH1 protein but has an N-terminal sequence that alters the localization of the OCH1 protein to or in the Golgi apparatus.

The wild type OCH1 gene of Pichia pastoris has an open reading frame that encodes a protein of 404 amino acids (SEQ ID NO: 2). Like other fungal Golgi glycosyltransferases, the Pichia pastoris OCH1 protein is a type II membrane protein, has a short cytoplasmic tail (Met1 to Tyr21 (SEQ ID NO: 25), or Ala2 to Tyr21), a membrane anchor domain (Phe22 to Ser44, i.e., FYMAIFAVSVICVLYGPSQQLSS (SEQ ID NO: 89)), a stem region, and a large C-terminal region containing the catalytic domain. See, e.g., Kim et al., J. Biol. Chem. 281:6261-6272 (2006); Nakayama et al., EMBO 11(7): 2511-2519 (1992); and Tu et al., Cell. Mol. Life Sci. 67:29-41 (2010).

The wild type OCH1 protein is generally localized in cis-Golgi. Golgi localization of the wild type OCH1 protein is believed to be dictated by the N-terminal region consisting of the cytoplasmic tail, the membrane anchor domain, and the stem region. In particular, the membrane anchor domain, including its amino acid constituents and length, plays an important role in the Golgi targeting of the protein. See, e.g., Tu et al. (supra).

The mutant OCH1 protein of this disclosure has an N-terminal sequence that alters the Golgi localization of the mutant OCH1 protein, as compared to the wild type OCH1 protein. As a result of this altered N-terminal sequence, the mutant OCH1 protein is either not properly targeted to or retained within the Golgi apparatus, or not properly targeted to or retained within the correct compartment within Golgi. The term “targeting” is meant the biological mechanisms by which proteins are transported to the appropriate destinations in the cell or outside of the cell. In specific embodiments, the mutant OCH1 protein of this disclosure lacks an N-terminal sequence that allows the Golgi targeting of the mutant OCH1 protein, such that the mutant OCH1 protein is not targeted the Golgi apparatus and is transported to another cellular location or secreted to outside of the cell.

In some embodiments, the alteration in the N-terminal sequence is a result of a mutation, i.e., addition, deletion or substitution, of one or more amino acids in the membrane anchor domain of the OCH1 protein. In specific embodiments, one or more amino acids in the membrane anchor domain have been deleted. In particular embodiments, at least 2, 3, 4, 5, 6, 7 or more amino acids, contiguous or otherwise, of the membrane anchor domain have been deleted. For example, some or all of the first 5 amino acids (FYMAI, SEQ ID NO: 90) of the membrane anchor domain are deleted.

In other embodiments, the alteration in the N-terminal sequence is a result of a mutation, i.e., addition, deletion or substitution, of one or more amino acids in the cytoplasmic tail of the OCH1 protein. In specific embodiments, one or more amino acids in the cytoplasmic tail have been deleted; for example, at least 2, 3, 4, 5, 6, 7 or more amino acids, contiguous or otherwise, of the cytoplasmic tail have been deleted. Examples of deletions in the cytoplasmic tail are found in Table 3. In other embodiments, deletion of one or more amino acids is combined with addition of one or more amino acids in the cytoplasmic tail.

In still other embodiments, the alteration in the N-terminal sequence is a result of a mutation of one or more amino acids in the stem region of the OCH1 protein; for example a deletion of one or more amino acids in the first 10, 20, 30, 40, 50, or 60 amino acids immediately following the membrane anchor domain.

In certain embodiments, the alteration in the N-terminal sequence is a result of a combination of mutations in the cytoplasmic tail, the membrane anchor domain, and/or the stem region of the OCH1 protein.

In specific embodiments, the alteration in the N-terminal sequence is a result of a combination of mutations in the cytoplasmic tail and the membrane anchor domain. For example, one or more amino acids in the cytoplasmic tail and one or more amino acids in the membrane anchor domain have been deleted. Examples of deletions in the N-terminal region of the OCH1 protein are listed in Table 3.

In other embodiments, in addition to deletions in one or more domains, one or more amino acids are added to the N-terminus of the protein, as long as the resulting mutant N-terminal sequence still fails to properly target or localize the OCH1 protein in Golgi. For example, the resulting mutant N-terminal sequence still lacks a functional membrane anchor domain. Whether a mutant sequence includes a membrane anchor domain can be readily determined based on the amino acid compositions and length. The membrane anchor domain of Golgi glycosyltransferases typically consists of 16-20 amino acids, which are hydrophobic and often contain aromatic amino acids, and has hydrophilic, often positively charged amino acids immediately outside both ends of the membrane span. See, e.g., Nakayama et al. (1992), supra. One example of a mutant OCH1 protein is set forth in SEQ ID NO: 3, which has its first 10 amino acids in place of the first 26 amino acids of the wild type OCH1 protein.

The mutant OCH1 protein disclosed herein contains a catalytic domain substantially identical to that of the wild type OCH1 protein.

The catalytic domain of the wild type OCH1 protein is located within the C-terminal fragment of 360 amino acids (i.e., within amino acids 45 to 404 of SEQ ID NO: 2). In some embodiments, the mutant OCH1 protein comprises a C-terminal fragment that is substantially identical to amino acids 45-404, 55-404, 65-404, 75-404, 85-404, 95-404, or 105-404 of SEQ ID NO: 2. By “substantially identical” it is meant that the sequences, when aligned in their full lengths, are at least 90%, 95%, 98%, 99%, or greater, identical. In most embodiments, the catalytic domain of the mutant OCH1 protein does not differ from the wild type domain by more than 10 amino acids, 8 amino acids, 5 amino acids, 3 amino acids, or 2 amino acids. In specific embodiments, the catalytic domain of the mutant OCH1 protein is identical with that of the wild type OCH1 protein. When one or more amino acids are different, it is preferable that the differences represent conservative amino acid substitutions. Examples of conservative substitutions include the substitution of a non-polar (hydrophobic) residue such as I, V, L or M for another; the substitution of one polar (hydrophilic) residue for another polar residue, such as R for K, Q for N, G for S, or vice versa; and the substitution of a basic residue such as K, R or H for another or the substitution of one acidic residue such as D or E for another.

The mutant OCH1 protein also substantially retains the catalytic activity of the wild type OCH1 protein, i.e., at least about 75%, 80%, 85%, 90%, 95% or more, of the □-1,6-mannosyltransferase activity of the wild type OCH1 protein. The activity of a particular OCH1 mutant protein can also be readily determined using in vitro or in vivo assays known in the art. See, e.g., Nakayama (1992), supra.

As described above, the strains of this invention include a mutant OCH1 allele which is transcribed into an mRNA coding for a mutant OCH1 protein, and do not include any other OCH1 allele that produces an mRNA coding for a functional OCH1 protein. Such strains can be engineered by a variety of means.

In some embodiments, the wild type OCH1 allele at the OCH1 locus on the chromosome of a Pichia pastoris strain has been modified or mutated to provide a mutant OCH1 allele (as illustrated in the Examples hereinbelow), or has been replaced by a mutant OCH1 allele (e.g., through homologous recombination). The modifications should be such that the resulting strain is stable with respect to the mutant OCH1 allele. That is, the mutant allele is maintained in the strain through generations (e.g., at least 10, 20, 30, 40, 50 or more cell divisions) suitable for both small volume flask culture and industrial size bioreactor culture, without reverting to an OCH1 allele coding for a functional OCH1 protein.

In other embodiments, a mutant OCH1 allele is introduced through an expression vector into a Pichia pastoris strain whose wild type OCH1 allele(s) (wild type OCH1 “allele” if haploid, or wild type OCH1 “alleles” if diploid) has already been disrupted hence no functional OCH1 protein is produced from the native OCH1 allele or native OCH1 locus. The expression vector can be an integrative vector designed to integrate the mutant OCH1 allele into the host genome; or a replicative vector (e.g., a plasmid) which replicates in the strain independent of the chromosomes.

Whether the mutant OCH1 allele is created at the native OCH1 locus by mutating or replacing the wild type OCH1 allele, or is provided via an expression vector in a strain whose wild type OCH1 allele(s) (wild type OCH1 “allele” if haploid, or wild type OCH1 “alleles” if diploid) has already been disrupted, it is important that the resulting mutant strain does not produce functional OCH1 protein through generations (e.g., at least 10, 20, 30, 40, 50 or more cell divisions). By “functional OCH1 protein” it is meant the wild type OCH1 protein or a functional equivalent of the wild type OCH1 protein, i.e., a protein that is targeted to Golgi and substantially retains the catalytic activity of the wild type OCH1 protein (i.e., at least about 80%, 85%, 90%, 95% or more, of the □-1,6-mannosyltransferase activity of the wild type OCH1 protein). To avoid reversion, homologous sequences in the strain should be removed to avoid homologous recombination which generates a wild type OCH1 allele.

The mutant OCH1 allele, whether present on the host chromosome or on an extra-chromosomal vector, is transcribed into mRNA. In other words, the strain is engineered such that the coding sequence of the mutant OCH1 allele is operably linked to a promoter to effect transcription. The promoter can be an endogenous promoter, such as the endogenous OCH1 promoter, a promoter heterologous to the OCH1 allele (e.g., an AOX1 promoter, a GAP promoter), and the like; or can be an exogenous promoter functional in Pichia pastoris. The level of transcription can be the same as, higher or lower than, the level of transcription of the wild type OCH1 allele in an unmodified Pichia pastoris strain (such as GS115).

Pichia pastoris strains having the genetic modifications to the OCH1 allele(s) described above include both haploid strains and diploid strains. For diploid strains having an OCH1 mutant allele integrated into a host chromosome, the strains can be either homozygous or heterozygous for the OCH1 mutant allele.

Pichia pastoris strains having the genetic modifications to the OCH1 allele(s) described above are robust and stable, and produce proteins with substantially homogeneous N-glycan structures with Man8GlcNAc2 being the predominant N-glycan.

### Genetic Engineering—A Nucleic Acid Coding for and Expressing an α-1,2-Mannosidase

In addition to the genetic modifications to the OCH1 allele(s) described above, the strains can be engineered to include a nucleic acid molecule which codes for and is capable of expressing an α-1,2-mannosidase or a functional fragment thereof which converts Man₈GlcNA_{c2} to Man₅GlcNA_{c2}, thereby providing Man₅GlcNA_{c2} as the predominant N-glycan form.

α-1,2-mannosidase (MS-I) is a well characterized family of enzymes. Most MS-I enzymes are known to be localized in the Golgi or endoplasmic reticulum, although a few are secreted and have extracellular activity. See, Gonzalez et al., Mol Biol Evolution 17:292-300 (2000). The topology of those enzymes that localize to the ER and the Golgi generally includes a luminal catalytic domain and an N-terminal transmembrane region. See, Herscovics, Biochimie 8: 757-62 (2001). The N-terminal region is composed of a stem region (closest to the luminal catalytic domain), a transmembrane domain, and a cytoplasmic tail. In the secreted MS-I enzymes, the extra-catalytic transmembrane region is also known as a leader sequence, serving as a signal for secretion of the enzyme. Detailed characterizations of various α-1,2-mannosidases can be found in Becker et al. (European J. Cell Biol 79: 986-992 (2000)) which studied the MS-I enzymes from mouse and S. cerevisiae and their catalytic domains; Schneikert and Herscovics (Glycobiology 4: 445-450 (1994)) which characterized the catalytic activity of a murine MS-I and its catalytic domain; Gonzalez et al. (J. Biol Chem 274: 21375-86 (1999)) which examined the activities and domains of several MS-I enzymes, including two enzymes from C. elegans, a human MS-I and the S. cerevisiae MS-I (from the ER); and Maras et al. (J. Biotechnology 77:255-263 (2000)), which characterizes the T. reesei α-1,2-mannosidase as belonging to the category of secretory MS-I's, which are composed of a catalytic domain and an N-terminal leader sequence.

The nucleic acid molecule encoding an α 1,2-mannosidase or a functional fragment thereof can derive from any species for use in this invention, including but not limited to mammalian genes encoding, e.g., murine α-1,2-mannosidase (Herscovics et al. J. Biol. Chem. 269: 9864-9871, 1994), rabbit α-1,2-mannosidase (Lal et al. J. Biol. Chem. 269: 9872-9881, 1994), or human α-1,2-mannosidase (Tremblay et al. Glycobiology 8: 585-595, 1998), fungal genes encoding, e.g., Aspergillus α-1,2-mannosidase (msdS gene), Trichoderma reesei α-1,2-mannosidase (Maras et al., J. Biotechnol. 77: 255-263, 2000), or a Saccharomyces cerevisiae α-1,2-mannosidase, as well as other genes such as those from C. elegans (GenBank Accession Nos. CAA98114 and CAB01415) and Drosophila melanogaster (GenBank Accession No. AAF46570) (see, e.g., Nett et al., Yeast 28:237-252, 2011, incorporated herein by reference).

By “functional part” or “enzymatically active fragment” of an α-1,2-mannosidase, it is meant a polypeptide fragment of a naturally occurring or wild type α-1,2-mannosidase which substantially retains the enzymatic activity of the full-length protein. By “substantially” in this context it is meant at least about 75%, 80%, 85%, 90%, 95% or more, of the enzymatic activity of the full-length protein is retained. For example, the catalytic domain of an α-1,2-mannosidase, absent of any N-terminal transmembrane or signal sequence, constitutes a “functional fragment” of the α-1,2-mannosidase. Those skilled in the art can readily identify and make functional fragments of an α-1,2-mannosidase based on information available in the art and a combination of techniques known in the art. The activity of a particular polypeptide fragment can also be verified using in vitro or in vivo assays known in the art.

In some embodiments, the nucleotide sequence coding for an α-1,2-mannosidase or a functional fragment is derived from the Trichoderma reesei α-1,2-mannosidase coding sequence. In specific embodiments, the nucleotide sequence codes for the Trichoderma reesei α-1,2-mannosidase described by Maras et al. J. Biotechnol. 77: 255-63 (2000), or a functional fragment thereof (such as the C-terminal catalytic domain of the full length protein).

In most embodiments, the strains are engineered such that the α-1,2-mannosidase or a functional fragment are targeted to the ER. In specific embodiments, the ER-targeting is achieved by including an ER-targeting sequence in the α-1,2-mannosidase or a functional fragment. Examples of ER-targeting sequences, i.e., sequences that target a protein to the ER so that the protein is localized or retained in the ER, include an N-terminal fragment of S. cerevisiae SEC12, an N-terminal sequence of S. cerevisiae α-glucosidase I encoded by GLS1, and an N-terminal fragment of S. cerevisiae α-1,2-mannosidase encoded by MNS1. See, also, Nett et al. (2011), supra. In a specific embodiment, the α-1,2-mannosidase or a functional fragment is targeted to the ER by including an ER-retention signal, HDEL (SEQ ID NO: 91), at the C-terminal of the α-1,2-mannosidase or its functional fragment.

The nucleic acid coding for an α-1,2-mannosidase or a functional fragment can be introduced through an expression vector into a Pichia pastoris strain. The expression vector can be an integrative vector designed to integrate α-1,2-mannosidase coding sequence into the host genome; or a replicative vector (e.g., a plasmid) which replicates in the strain independent of the chromosomes. In cases of an integrative vector, the vector can be designed to achieve integration of the nucleic acid into the wild type OCH1 allele (e.g., through single or double cross over homologous recombination) and simultaneous disruption of the wild type OCH1 allele.

### SuperM5 Strains

This disclosure provides Pichia pastoris strains that are robust, stable, and transformable, and produce proteins with substantially homogeneous Man5GlcNAc2 N-glycans. These strains are also referred to herein as SuperM5 or SuperMan5 strains.

SuperM5 strains are genetically engineered to include a mutant OCH1 allele which is transcribed into an mRNA coding for a mutant OCH1protein that contains a catalytic domain substantially identical to that of the wild type OCH1 protein, but lacks an N-terminal sequence necessary to target the OCH1 protein to the Golgi apparatus. The strains do not include any other OCH1 allele that produces an mRNA coding for a functional OCH1 protein. The strains are additionally genetically engineered to contain a nucleic acid coding for and expressing an α-1,2-mannosidase or a functional fragment thereof, which is targeted to the ER and converts Man8GlcNAc2 to Man5GlcNAc2.

An example of a SuperM5 strain is described in Example 1. The nucleotide sequence of the OCH1 locus of this strain is set forth in Table 1 and SEQ ID NO: 1. Constructed using the M5-Blast strain described in Jacobs et al. (2009), the SuperM5 strain is superior over M5-Blast in terms of robust growth, stability, and homogeneity of the M5 glycans produced.

### Genetic Engineering—Introduction of Additional Enzymes

The strains can be additionally modified to express other, downstream enzymes (or functional fragments thereof) in the glycosylation pathway towards making hybrid- and complex-type N-glycans. Such additional enzymes include, e.g., one or more of GlcNAc transferase I (GnT-I), β-1,4-galactosyltransferase 1 (GalT), mannosidase II (Man-II), and GnT-II, among others. See Jacobs et al. (2009); U.S. Pat. No. 7,029,872 to Gerngross.

GnT-I catalyzes the addition of a α-1,2-linked GlcNAc residue to the α-1,3-mannose of the trimannosyl core in Man5GlcNAc2. Introduction of the GnT-I activity can be achieved by transforming with a vector comprising a nucleic acid sequence coding for a GlcNAc-transferase I (GnT-I) for use in this invention. Such nucleic acid sequence can derive from any species, e.g., rabbit, rat, human, plants, insects, nematodes and protozoa such as Leishmania tarentolae. In specific embodiments, the nucleotide sequence encodes a human GnT-I. The GnT-I or a functional part thereof is targeted to the Golgi apparatus, which can be achieved by including a yeast Golgi localization signal in the GnT-I protein or a functional part thereof. In certain embodiments, the catalytic domain of human GnT-I is fused to the N-terminal domain of S. cerevisiae Kre2p, a glycosyltransferase with a known cis/medial Golgi localization.

GalT catalyzes the addition of a galactose residue in β-1,4-linkage to the β-1,2-GlcNAc, using UDP-Gal as donor substrate. Introduction of the GalT activity can be achieved by transforming with a vector comprising a nucleic acid sequence coding for a GalT or a functional fragment thereof, which can derive from human, plants (e.g. Arabidopsis thaliana), insects (e.g. Drosophila melanogaster). The GalT or a functional part thereof is genetically engineered to contain a Golgi-retention signal and is targeted to the Golgi apparatus. An exemplary Golgi-retention signal is composed of the first 100 amino acids of the Saccharomyces cerevisiae Kre2 protein.

Man-II acts to remove both terminal α-1,3- and α-1,6-mannoses from GlcNAcMan₅GlcNAc₂ N-glycans. The presence of a terminal β-1,2-linked GlcNAc residue on the α-1,3-arm is essential for this activity. Introduction of the Man-II activity can be achieved by transforming a strain with a nucleic acid vector coding for a Man-II protein or a functional fragment thereof, engineered to contain a Golgi-localization signal. As an example, a suitable nucleic acid can encode the catalytic domain of Drosophila melanogaster Man-II, fused in frame to the Golgi-localization domain of S. cerevisiae Mnn2p.

GnT-II catalyzes the addition of a second β-1,2-linked GlcNAc residue to the free α-1,6-mannose of the trimannosyl core. Introduction of the GnT-II activity can be achieved by transforming with a vector which contains a nucleotide sequence coding for a GnT-II protein or a functional fragment thereof. GnT-II genes have been cloned from a number of species including mammalian species and can be used in the present invention. As an example, a suitable nucleotide sequence codes for the catalytic domain of rat GnT-II fused to the N-terminal part of S. cerevisiae Mnn2p.

### Other Manipulations to the Strains

The strains disclose herein can include additional features, achieved by various suitable manipulations (such as cross or recombinant engineering), including, e.g., having a mutant auxotroph gene (e.g., his−) to facilitate cloning and selection, having protease deficiency for limiting product degradation (e.g., pep4−, prb1−, and/or sub2−), having a slow methanol utilization phenotype (e.g., mutS).

In specific embodiments, this disclosure provides the following strains:
SuperMan5, P. pastoris, och1−, blasticidin resistant, Mannosidase I from T. reesei (=His+);
SuperMan5 (his−), P. pastoris, och1−, his4−, blasticidin resistant, Mannosidase I from T. reesei;
SuperMan5 (mutS), P. pastoris, och1−, blasticidin resistant, Mannosidase I from T. reesei (slow methanol utilization);
SuperMan5 (pep4−), P. pastoris, och1−, blasticidin resistant, Mannosidase I from T. reesei (protease deficient);
SuperMan5 (prb1−), P. pastoris, och1−, blasticidin resistant, Mannosidase I from T. reesei (protease deficient);
SuperMan5 (pep4−, sub2−), P. pastoris, och1−, blasticidin resistant, Mannosidase I from T. reesei (protease deficient);
SuperMan5 (pep4−, prb1−), P. pastoris, och1−, blasticidin resistant, Mannosidase I from T. ressei (protease deficient).

### Use of the Strains

A heterologous protein with one or more N-glycosylation sites can be expressed in the strains of this invention by transforming a strain of this invention with an expression vector coding for the heterologous protein, to obtain a preparation of the heterologous protein substantially homogeneous in its N-glycan structures.

### Example 1—Generation of a SuperM5 Strain

This Example describes the creation of a SuperM5 strain from a M5-Blast strain described in Jacobs et al. (2009), Nature Protocols 4:58-70 (incorporated herein by reference).

The M5-Blast strain is a modification of the P. pastoris GS115 strain wherein the endogenous mannosyltransferase gene OCH1 is disrupted by the insertion of a vector comprising an α-1,2 mannosidase gene (pGlycoSwitchM5-Blast vector) through single crossover homologous recombination. As a result of the single crossover homologous recombination, the integrated mannosidase expression cassette is flanked by approximately 450 bp of homologous sequences from the OCH1 ORF. The sequence of the OCH1 genomic locus of this M5-Blast strain is set forth in SEQ ID NO: 53. Sequencing revealed the loss of 10 bp at the junction between the pGlycoSwitchM5-Blast vector sequence and the OCH1 ORF 3′ fragment, resulting in the loss of one of the three desired stop codons from pGlycoSwitchM5-Blast vector upstream of the OCH1 C-terminal fragment, and frame shifted the second and third stop codons to a different reading frame than the fragment. As a result, the actual ORF was extended 28 bp upstream to an in-frame ATG codon in the vector backbone. Phe27 of the wild type protein became Phe11 of the new ORF, and the new predicted signal sequence consists partially of the old signal anchor and new, fused sequence from the vector backbone. The amino acid sequence of this new ORF is set forth in SEQ ID NO: 3 (with the first 25 amino acids being the predicted new signal sequence).

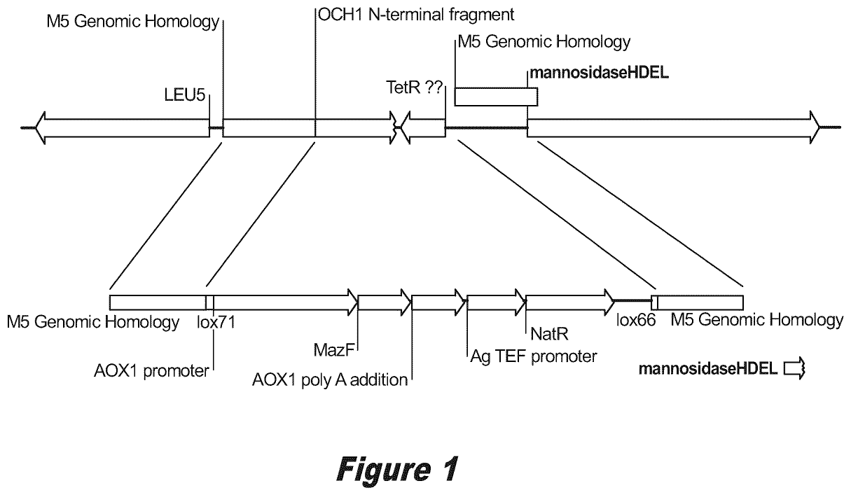

The N-terminal region of the OCH1 genomic locus after the single crossover homologous recombination event is diagrammed in FIG. 1, along with the construct used to remove this N-terminal region by double crossover homologous recombination. The construct contained both selection and counter-selection markers flanked by a lox71-lox66 pair, allowing for subsequent removal of the selection/counter-selection cassette by cre mediated recombination. The sequence of the double crossover selection/counter-selection cassette with homology arms is set forth in SEQ ID NO: 58, and its creation is described below in this Example.

In order to confirm the sequence of the targeted region prior to creating the cross-over construct, PCR primers were designed to amplify ˜1650 bp of DNA encompassing the region upstream of the mannosidase ORF. Using Phusion polymerase (NEB), PCR primers 80670918 and 80670919 amplified an appropriate sized fragment from M5-Blast genomic DNA. The PCR product was TOPO cloned and sequence verified. The DNA sequence demonstrated that the mannosidase expression vector had integrated into the GS115 genome correctly at this end of the insertion.

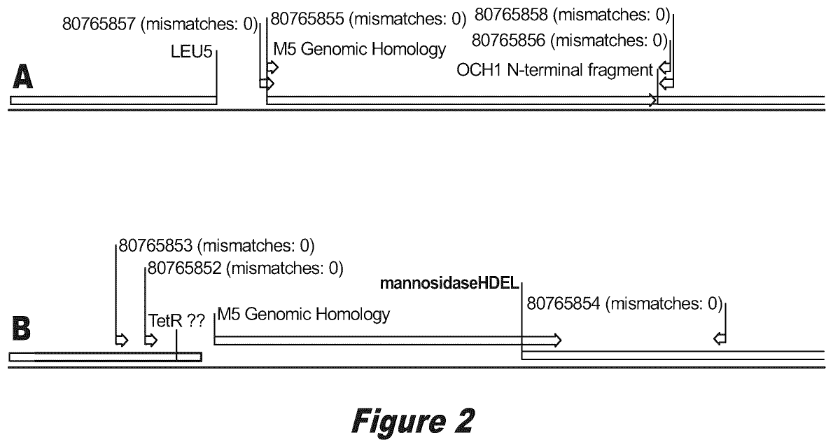

Flanking PCR primers were designed to amplify the homology regions shown in FIG. 1 from M5-Blast genomic DNA. The alignment of these PCR primers is shown in FIG. 2. Use of Phusion polymerase resulted in successful PCR reactions from M5-Blast genomic DNA.

PCR products for the following primer pair combinations were gel isolated and used as templates for the addition of lox71 and lox66 recombination sites:
80765855-80765856 (642 bp, FIG. 2A)
80765857-80765858 (658 bp, FIG. 2A)
80765852-80765854 (910 bp, FIG. 2B)
80765853-80765854 (956 bp, FIG. 2B)

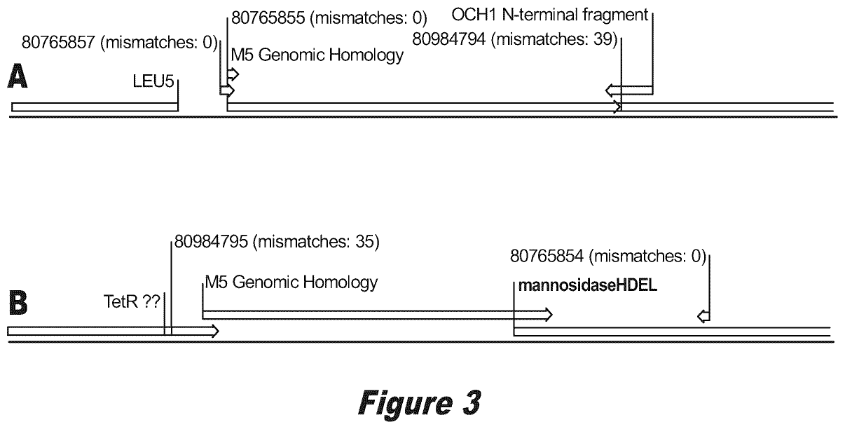

Mismatch PCR primers were designed to add the lox sites at the appropriate ends of the two homology arms. These mismatch primers are diagrammed in FIG. 3. PCR reactions with Phusion polymerase were successful in generating the correct sized DNA products from each of the 3 reactions:
80765855-80984794 (670 bp, FIG. 3A)
80765857-80984794 (681 bp, FIG. 3A)
80984795-80765854 (850 bp, FIG. 3B)

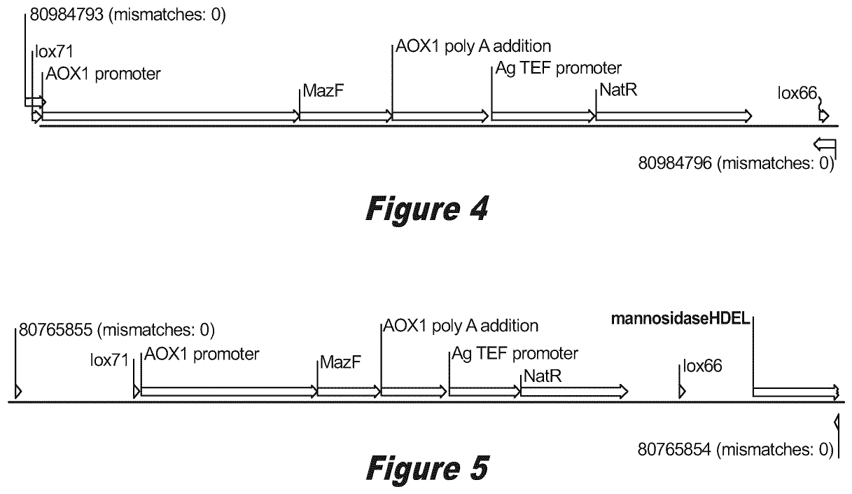

In addition to adding lox sites to the arms, PCR primers were designed to add appropriate M5-Blast Pichia genomic DNA extensions onto an existing lox71-MazF-Nat^{R}-lox66 cassette. Again, Phusion polymerase was used to generate the correct PCR product, as shown in FIG. 4. The primer pair used:
80984793-80984796 (2941 bp, FIG. 4)

The PCR product of the selection/counter-selection cassette was gel purified and a three piece overlap PCR was performed to attach the homology arms to the cassette. Briefly, the three pieces were cycled 20× in the absence of primers to anneal and extend the overlap at the ends of the fragments. The cycled mix was then diluted and cycled 35× in the presence of the primers diagrammed in FIG. 5.

The PCR reaction was performed with Phusion polymerase, using an extension time of 3 min. Primers are detailed below:
80765855-80765854 (4311 bp, FIG. 5)

This PCR product was gel isolated and TOPO cloned. Selection of the TOPO cloning was performed on LB-Nat plates to ensure the inclusion of the selection cassette. DNA sequencing was performed on multiple isolates to determine the homology arm sequences. The final isolate contained a functional Nat^{R} expression cassette, the lox71 and lox66 recombination sites and the correct homology arms.

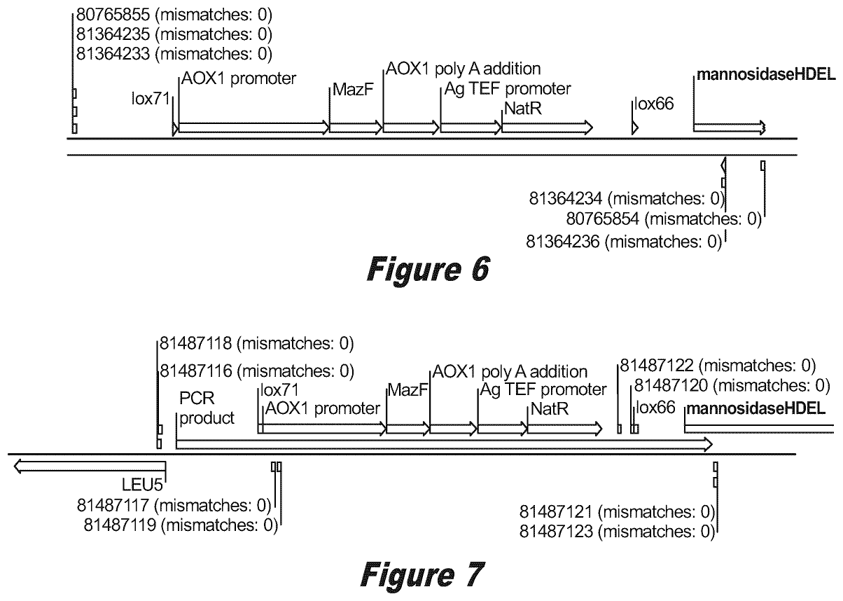

PCR primers internal to the cloned fragment detailed in FIG. 5 were used to generate linear DNA for Pichia pastoris transformation. Two independent sets of primers were designed:
81364233-81364234 (4063 bp, FIG. 6)
81364235-81364236 (4060 bp, FIG. 6)

PCR reactions were performed using Phusion polymerase with an extension time of 100 sec.

The PCR products were purified by agarose gel electrophoresis and eluted from the binding matrix with water. The M5-Blast Pichia pastoris strain was made competent for electroporation using a standard DTT/sorbitol treatment. Electroporation was performed using 1 mm cuvettes containing 20 μl competent cells and 1-2 μl of purified linear DNA. Transformation mixtures were plated on YPD-Nat agar.

After electroporation, cells were grown out at 30° C. for 3 days. Individual transformants were patched to YPD-Nat for storage and analysis. FIG. 7 shows the theoretical arrangement of the OCH1 locus after proper double crossover integration of the PCR product(s) into the M5-Blast genome. PCR primer pairs were designed to check that the nourseothricin-resistant isolates were the result of homologous recombination, rather than random integration of the PCR product(s) into the genome. These PCR primer pairs are diagrammed on FIG. 7.
81487116-81487117 (895 bp, FIG. 7)
81487118-81487119 (937 bp, FIG. 7)
81487120-81487121 (656 bp, FIG. 7)
81487122-81487123 (756 bp, FIG. 7)

A total of 24 independent isolates were screened by PCR and 2 isolates that appeared correct were further characterized by DNA sequencing of the PCR products. The two isolates were struck to single colonies on YPD medium and retested on YPD-Nat. Small scale genomic DNA preparations were made using phenol/chloroform glass bead lysis. Based on the sequencing results of the 81487116-81487117, 81487118-81487119, 81487120-81487121 and 81487122-81487123 primer pairs on these genomic extracts, both isolates contained the lox71-lox66 selection/counter-selection cassette at the proper location in the M5-Blast genome. There were no mutations introduced by the initial PCR reaction to generate the transformation fragment, the recombination junctions at both ends were identical to M5-Blast “wild-type” DNA sequence, and both the lox71 and lox66 sites were intact. The DNA sequence of the OCH1 locus after double cross over recombination is set forth in SEQ ID NO: 59.

The two isolates (A1-2 and A4-3) were transformed with a plasmid constitutively expressing cre recombinase. Briefly, both strains were made electro-competent using a DTT/sorbitol procedure, electroporated with circular plasmid and plated on YPD-G418. Transformants were grown out at 30° C. for several days and colonies picked. Colonies were either transferred directly to methanol plates to induce the MazF counter-selection or patched to YPD to allow loss of the cre-ARS plasmid prior to MazF induction. Methanol induction was carried out on both BMMY (1% methanol) and CSM (complete synthetic medium, 0.5% methanol). Plates were supplemented with methanol daily by adding 100 μl methanol to the inverted plate lid. Incubation was carried out at 30° C. There was significant colony formation under all conditions tested; growth on methanol appeared independent of whether the transformant came directly from YPD-G418 or had undergone an intermediate patching on YPD without G418.

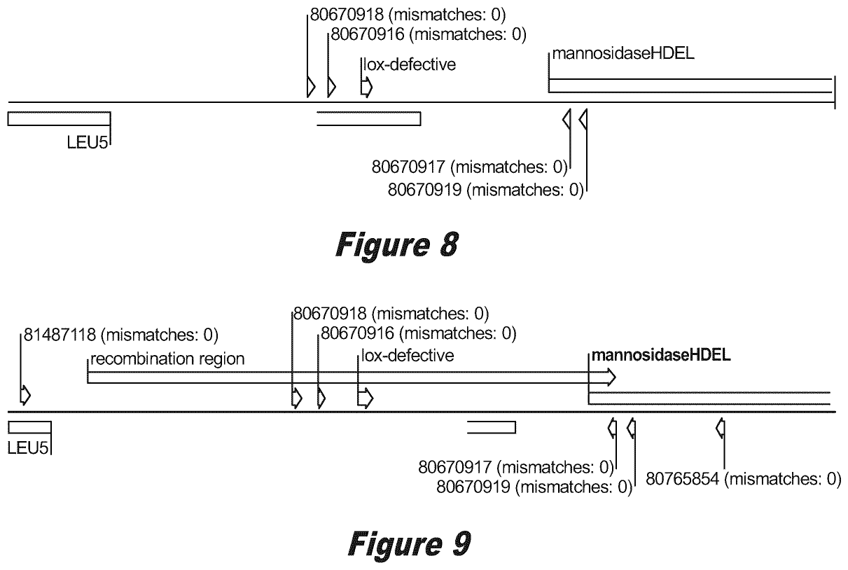

Cre recombination should remove the DNA sequences between the lox71 and lox66 sites, leaving only a defective lox site scar in the genome. The theoretical result of this recombination event is shown in FIG. 8. PCR primers were designed to amplify the region containing the defective lox scar. Twenty colonies that grew on methanol were screened by PCR to determine the loss of the selection/counter-selection cassette. PCR primers used were:
80670916-80670917 (680 bp, FIG. 8)
80670918-80670919 (782 bp, FIG. 8)

Seventeen of twenty isolates generated the appropriate PCR product with the first primer pair. Most, but not all, of the 17 also showed an appropriate product with the second primer pair. Each of the 17 isolates was patched to YPD, YPD-Blast, YPD-Nat and YPD-G418 to test for the presence or absence of the drug selection markers. If the cre plasmid had properly removed the selection/counter-selection cassette and subsequently been lost, the resulting strain should be blasticidin resistant and sensitive to both G418 and nourseothricin. All isolates were blasticidin resistant and nourseothricin sensitive. A few retained G418 resistance (still contained the cre plasmid, perhaps integrated) and were discarded. Of the remainder, 4 were picked for DNA sequencing of the LEU5-mannosidaseHDEL intergenic region.

Existing PCR primers were used to amplify the genomic region spanning LEU5 and the mannosidaseHDEL ORF.
81487118-80765854 (1602 bp, FIG. 9)

PCR amplification was performed using Phusion polymerase on genomic DNA that had been prepared by phenol/chloroform glass bead extraction. Multiple internal sequencing primers were used to verify the entire sequence of the 1602 bp PCR product. All 4 of the sequenced PCR products were correct, and contained a defective lox site at the proper location between the LEU5 gene and the mannosidaseHDEL ORF. Both the LEU5 promoter and the GAP promoter driving mannosidaseHDEL expression were intact and identical to the promoters present in the starting M5-Blast strain. The DNA sequence of the OCH1 locus after double crossover recombination and cre recombination is set forth in SEQ ID NO: 1.

Glycerol stocks of each of the 4 isolates (and 2 parental strains prior to cre recombination) were prepared.
bG yeast-100015 A1-2 (pre-recombination)
bG yeast-100016 A4-3 (pre-recombination)
bG yeast-100017 isolate 1 (post-recombination)
bG yeast-100018 isolate 2 (post-recombination)
bG yeast-100019 isolate 3 (post-recombination)
bG yeast-100020 isolate 4 (post-recombination)

Each glycerol stock was streaked and retested for the appropriate markers:
bG yeast-100015 his−, blasticidinR, nourseothricinR
bG yeast-100016 his−, blasticidinR, nourseothricinR
bG yeast-100017 his−, blasticidinR, nourseothricinS
bG yeast-100018 his−, blasticidinR, nourseothricinS
bG yeast-100019 his−, blasticidinR, nourseothricinS
bG yeast-100020 his−, blasticidinR, nourseothricinS

All glycerol stocks tested as expected.

YPD stabs of all 6 isolates were generated and subjected to glycoanalysis. Glycerol stock bG yeast-100017 was used to generate a large genomic DNA preparation for genomic sequencing. In addition, samples were prepared from wild-type GS115 and the M5-Blast strain. Briefly, cell pellets from 100 ml yeast cultures (YPD, 30° C. growth) were resuspended in 1 M sorbitol/100 mM citrate (pH 6.0) and treated with Zymolyase (Zymo Research) containing RNase for 2 h at 37° C. SDS was added to 0.5% to lyse spheroplasts. Proteinase K was then added and the mixture incubated at 50° C. overnight. An equal volume of phenol/chloroform was added and the mixture gently rocked for 30 min. After centrifugation, the upper aqueous layer was removed and DNA precipitated with isopropanol. The threaded DNA was spooled from the solution and resuspended in TE. The DNA was reprecipitated with ethanol and then washed with 70% ethanol, air-dried and resuspended a final time in TE.

DNA was distributed in multiple tubes:
bG DNA-100215 GS115 genomic DNA
bG DNA-100216 GS115 genomic DNA
bG DNA-100217 GS115 genomic DNA
bG DNA-100221 bG yeast-100017 genomic DNA
bG DNA-100222 bG yeast-100017 genomic DNA
bG DNA-100223 M5-Blast genomic DNA
bG DNA-100224 M5-Blast genomic DNA

In order to test the genomic DNA isolates and verify that the manipulations performed in creating the bG yeast-100017 strain had not altered the mutant form of the OCH1 ORF, the N-terminal region of the OCH1 ORF was isolated from bG DNA-100221 (new strain) and bG DNA-100223 (M5—Blast strain) by PCR and resequenced. Both DNA preparations were identical at the OCH1 ORF locus, and contained the 10 bp deletion as described above.

Primers used in this Example are listed below:

| SEQ ID | |
|---|---|
| 80 | CAAGTTGCGCCCCCTGGCA |
| 80 | TGGAGCAGCTAATGCGGAGGA |
| 80 | AGTTCCGCCGAGACTTCCCCA |
| 80 | TTCAGCCGGAATTTGTGCCGT |
| 80 | ATCCAGGGTGACGGTGCCGA |
| 80 | GCAAGAGGCCCGGCAGTACC |
| 80 | CCGCCCTCGTAGGGTTGGGAG |
| 80 | TTCGCGGTCGGGTCACACA |
| 80 | AACTGCCATCTGCCTTCGCC |
| 80 | CAAATCGCGGGTTCGCGGTC |
| 80 | GAGCAAACTGCCATCTGCCTTCG |
| 80 | GTGTTCGTAGCAAATATCATCAGCCTACCGTT |
| 98 | CGTATAGCATACATTATACGAAGTTATGGATC |
| 80 | TTTGGATGTTAGATCCATAACTTCGTATAATGT |
| 98 | ATGCTATACGAACGGTAGGCTGATGATATTTG |
| 80 | GCCGCCATCCAGTGTCATAACTTCGTATAGCA |
| 98 | TACATTATACGAACGGTACTTTTTTGTAGAAA |
| 80 | ACACCAAGACATTTCTACAAAAAAGTACCGTT |
| 98 | CGTATAATGTATGCTATACGAAGTTATGACAC |
| 81 | GTGTTCGTAGCAAATATCATCAGCCTACCG |
| 81 | ACACCAAGACATTTCTACAAAAAAGTACCGT |
| 81 | TTCGCGGTCGGGTCACACAC |
| 81 | GGAGCAGCTAATGCGGAGGATGC |
| 81 | CGGTCGGGTCACACACGGAG |
| 81 | TGGAGCAGCTAATGCGGAGGA |
| 81 | TGAGTCCTGGTGCTCCTGACG |
| 81 | CCCCTCCTGTTGCGTTTGGC |
| 81 | AGCGTTCTGAGTCCTGGTGCT |
| 81 | GGTCCTGCGTTTGCAACGGT |
| 81 | ACTAACGCCGCCATCCAGTGTC |
| 81 | GCTTCAGCCGGAATTTGTGCCG |
| 81 | CGCCTCGACATCATCTGCCC |
| 81 | TCAGCCGGAATTTGTGCCGT |

### Example 2—Storage and Handling

SuperM5 was stored in different conditions at −80° C., −4° C., 20° C. and at room temperature. Strains were stored as frozen glycerol stocks and as stab cultures. Different cultures were stored and thawed for different experiments and for shipping to collaborators for testing. In all cases the strains recovered, plated and cultured similar to the parent Pichia pastoris GS115 strain and grew in both complex and defined media similar to the parent strains. The SuperM5 strains transformed similarly as the parent strain and proteins were expressed with the mannose-5 glycosylation as the predominate glycoform, or the only glycoform. Strains have been repeatedly stored and regrown to establish robustness of the SuperM5 strains.

### Example 3—Analysis of Test Proteins in P. pastoris Strains

The genes for Candida antartica lipases A and B, human transferrin, and the human CH2 domain from IgG were integrated into the SuperM5 genome using standard transformation methods. In all cases significant amounts of protein were produced and secreted into the medium. Transformed strains and media-containing protein were tested for glycan analysis using previously published methods. In all cases, the glycan profiles for the test proteins and for the strain glycoproteins demonstrated a mannose-5 glycan structure with no other higher mannose structures detected by the methods used.

### Example 4—Analysis of Cell Wall Mannoproteins in P. pastoris Strains

Twelve Pichia pastoris strains and the Man5-Blast strain were started in a 24-well plate containing 2 ml YPD and grown overnight at 28° C. while shaking (250 rpm). After growth, cells were harvested by centrifugation (3000 g for 5 min at room temperature) and cell wall mannoproteins were extracted according to the protocol by Jacobs et al. (see Jacobs et al., 2009, Nature Protocols 4(1):58-70). The extracted mannoproteins (in 100 μl ddH20) were diluted to 300 μl with RCM buffer (8 M urea, 3.2 mM EDTA, 360 mM Tris-HCL, PH 8.6). N-glycans were prepared from these samples following the 96-well on-membrane deglycosylation procedure as published by Laroy et al. (Laroy et al., 2006, Nature Protocols, 1: 397-405).

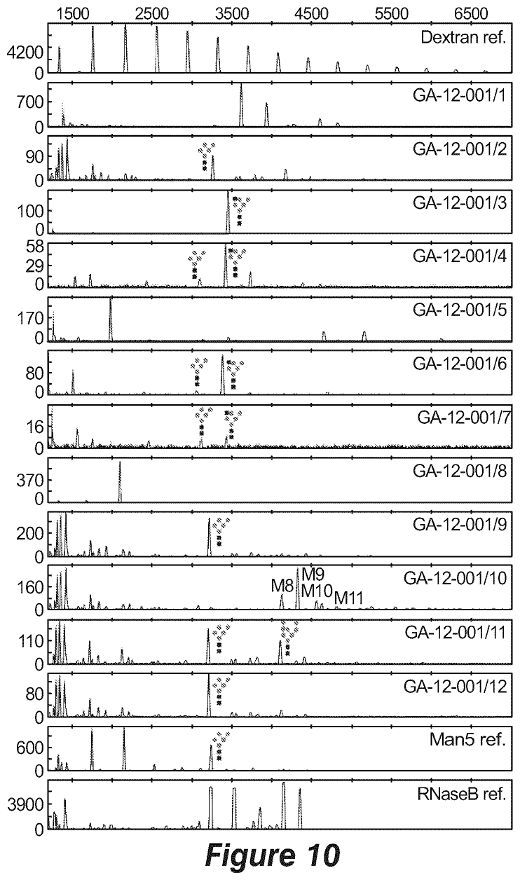

After labeling the dried N-glycans with 8-aminopyrene-1,3,6-trisulphonic acid2, the excess of label was removed using size exclusion chromatography (Sephadex G-10 resin2). The samples were finally reconstituted in 10 μl of ultrapure water and diluted 10× prior to their injection (80″ at 1.2 kV) in the capillaries (e.l. 36 cm; i.d. 50 μm) of an ABI 3130 DNA sequencer. The following settings were applied: Oven temperature: 60° C. Run voltage: 15 kV; Prerun voltage: 180″ Run time: 1000″; Prerun time: 15 kV. The Genemapper v3.7 was used to analyze the obtained data and structures were assigned to the peaks (see FIG. 10).

### Example 5—Materials and Methods

Below describes non-limiting examples of materials and methods for the present invention.

Plasmids and strains: Pichia pastoris expression vector pPICZaA was purchased from Invitrogen Corporation; pUC19/GM-CSF plasmid (containing GM-CSF Gene sequence) was synthesized by Shanghai Qing-Lan Biotech Co., Ltd.; Saccharomyces cerevisia expression vector pYES2, Pichia pastoris X-33 (wild Type), E. coli JM109 were from the inventors' laboratory.

Reagents and instruments: Taq DNA polymerase, Pfu DNA polymerase, restriction enzymes, T4 ligase, 5-fluoroorotic acid (5-FOA) was purchased from Shanghai Biological Engineering Technology Services Co., Ltd.; Zymolyase was purchased from Sigmag company (USA); N-glycosidase F (PNGase F) was purchased from New England Biolabs, Inc. (USA); peptone, yeast extract, yeast nitrogen base without amino acids (YNB) were purchased from BIO BASIC INC (Canada). PCR machine (PTC100) was from MJ Research, Inc. (USA); electrophoresis systems, gel imaging system were from Bio-Rad (USA); AKTA purification system purchased from GE (USA).

Primers: based on the reported Pichia URA3 (orotidine-5′-Phosphate decarboxylase) gene sequence (GenBank: AF321098), two pairs of extension amplification primers based on homologous fragment were designed: URA5F, URA5R and URA3F, URA3R; based on Saccharomyces cerevisiae expression vector pYES2 sequence, primers pYES2F and pYES2R were designed; based on the GenBank (E12456) reported Pichia OCH1 gene sequence, two pairs of amplification primers based homologous sequence were designed: OCH5F, OCH5R and OCH3F, OCH3R. The internal identification primers (in) 5F, (in) 3R were also based on the same sequence; universal primers 5′ AOX1, 3′ AOX1 sequences were based on references. Primers were synthesized by Shanghai Biological Engineering Technology Services Co., Ltd.

Yeast cell culture, genomic extraction and PCR conditions were performed based on known protocols.

The construction of URA3 homologous replacement DNA sequence: using the X-33 strain genome as a template and primer pairs URA5F, URA5R and URA3F, URA3R, the homologous fragments of both sides of URA3 genes, URA5′ and URA3′, a 700 bp and a 600 bp, respectively, were PCR amplified. Then using URA5′ and URA3′ as templates and URA5F and URA3R as a primer pair, the URA5-3, the target homologous replacement DNA fragment for URA3 gene was PCR amplified, which was about 1300 bp in size.

The construction of pYXZ plasmid: using plasmid pYES2 as a template and primer pair pYES2F and pYES2R, the sequence that contains URA3 gene was PCR amplified. The PCR product was purified and digested with Sal I and followed with ligation reaction. The self-ligased plasmid pYXZ was transformed into E. coli JM109, and plated on LB plates containing ampicillin to select positive clones.

The cloning of OCH1 homologous arm: using the X-33 strain genome as a template and primer pairs OCH5F, OCH5R and OCH3F, OCH3R, to PCR amplify the 5′ and 3′ ends of the OCH1 gene homologous arms, OCH5′ and OCH3′ and its fusion fragment OCH3-5. The method used was similar to what has been described above. The fragment sizes were 1000 bp, 700 bp and 1700 bp, respectively.

The construction of Knockout plasmid pYXZ-OCH1: the inventors digested the OCH1 gene 5′ and 3′ homologous fusion fragment OCH3-5 with Nhe I and Sal I and cloned the fragment into pYXZ plasmid digested with Sal I and Nhe I to make the knockout plasmid pYXZ-OCH1.

Knockout the URA3 gene from Pichia pastoris X-33 to construct auxotrophic selection marker: X-33 competent cells were shock transformed using the fusion fragment URA5-3 arm that has homologous sequence to both ends of the URA3 gene; the transformed cells were spread on MD medium containing 5-FOA and uracil (YNB 1. 34%, glucose 2%, agar 1.5%, uracil 100 μg/mL, 5-FOA 1 mg/mL), and incubated at 30 degrees Celsius for 3-5 days. Single colonies grown on the medium were selected and seeded with a toothpick, respectively, to MD medium (YNB 1. 34%, glucose 2%, agar 1.5%) and MDU medium (YNB 1. 34%, glucose 2%, agar 1.5%, uracil 100 μg/mL), and incubated at 30 degrees Celsius for 3-5 days. Then, strains that grew well on the MDU medium but could not grow on the MD medium were selected. The selection process was repeated for 3 rounds to get stable traits and the final strains were confirmed by PCR reaction using URA5F, URA3R as primers and genomic DNA as template.

OCH1 gene knockout of Pichia pastoris X-33: the knockout plasmid pYXZ-OCH1 was linearized at Mlu I site that is located between the two homologous arms and electric shock transformed into the X-33 (ura3-) competent cells, and spread on MD medium, and incubated at 25 degrees Celsius for about a week. Single colonies were picked with a toothpick and seeded to the same coordination on two plates with YPD medium (peptone 2%, yeast extract 1%, glucose 2%, agar 1.5%), and incubated at 25 degrees Celsius and 37 degrees Celsius, respectively for a few days. The colonies that grew well at 25 degrees Celsius but could not grow at 37 degrees Celsius were extracted to obtain genomic DNA. OCH1 gene external primers OCH5F, OCH3R and internal primers (in) 5F, (in) 3R were used for PCR identification.

Construction of expression vector: the plasmid pUC19/GM-CSF from the inventors' own laboratory was double digested with EcoRI and Not I. The GM-CSF gene fragment was extracted (a 6×His tag sequence was introduced), and cloned into Pichia pastoris expression vector pPICZaA digested with the same restriction enzymes to make the expression vector pPICZaA/GM-CSF. Positive clones were selected and confirmed with restriction enzyme digestion and sequencing.

The expression and analysis of GM-CSF in Pichia pastoris X-33 and X-33 (och1-): linearize the expression vector pPICZαA/GM-CSF with Sal I and electrically shock transformed the plasmid into X-33 and X-33 (och1-) competent cells. Shock mixture was spread to culture cloth coated with YPDZ medium (each containing 100 μg/mL, 300 μg/mL, or 500 μg/mL Zeocin), the X-33 transformants were grown at 30 degrees Celsius for 3-5 days, and X-33 (och1-) transformants were cultured at 25 degrees Celsius for about a week. Single colonies that grew well were picked to extract genomic DNA and identified with PCR reaction using primers 5′AOX, 3′AOX1 to select positive transformants. Positive X-33/PICZαA/GM-CSF cells were inoculated into 2 mL of YPD medium (2% peptone, 1% yeast extract, 2% glucose), incubated at 30 degrees Celsius for 24 h. The culture was used to inoculate (5% inoculation ratio) into 10 mL of BMGY medium (2% peptone, yeast extract 1%, YNB 1. 34%, glycerol 2%, 100 mmol/L phosphate buffer, pH 6.0). After incubation at 30 degrees Celsius for 36 h, the culture was centrifuged to remove the supernatant and the pellet was resuspended to 3 mL of BMMY medium (yeast extract 1%, YNB 1.34%, peptone 2%, 100 mmol/L phosphate buffer, PH 6. 0), 2% methanol was added to induced expression: X-33 (och1-)/pPICZaA/GM-CSF positive cells were cultured in the YPD medium at 25 degrees Celsius for 48 h, BMGY at 25 degrees Celsius for 48 h, and induced expression at 25 degrees Celsius. Expression induction condition was same as that used in X-33 cells, methanol was added every 24 h and the induction was for 72 h. Once it was finished, the cell cultures were centrifuged and supernatant was collected for protein analysis.

### Example 6—Transcriptome Analysis of M5-Blast and SuperM5 Strains

Strain Growth For RNA Isolation. BG10, GS115, M5 Blast and SuperM5 (described in Example 1) strains were maintained on YPD Agar plates as patches. For transcriptome analysis, a 50 ml culture of each strain was inoculated from a patch and grown in BMGY at 30° C., 200 rpm for approximately 16 hours. The stationary culture was diluted 100-fold into fresh BMGY medium and grown at 30° C., 200 rpm for 6 hours. This time point was considered exponential growth with glycerol as the carbon source. Aliquots were spun down in 15 ml tubes, supernatants discarded and the cell pellets rapidly frozen in liquid nitrogen. Cell pellets were stored at −80° C. for subsequent total RNA isolation.

Total RNA Isolation. FastRNA SPIN kits (MP Bio) were used to isolate total RNA. Cell lysis was per-formed using a BioSpec Mini-Beadbeater 96. Total RNA was eluted from the spin column in 15 μl of RNase/DNase-free water, frozen in liquid nitrogen and stored at −80° C. RNA samples were shipped on dry ice for RNA-Seq analysis on an Illumina HiSeq machine. RNA samples were analyzed using an Agilent BioAnalyzer, and all showed intact yeast ribosomal RNA peaks.

RNA Library Generation and Sequencing. mRNA libraries were prepared using Illumina reagents. A TruSeq RNA Sample Preparation Kit was used to selectively generate bar-coded cDNA from polyA RNA. After bar-coding and amplification, a total of 12 samples were pooled (4 samples for this study) for analysis. Fifty base, single end reads were performed. Data was supplied to BioGrammatics in standard FASTQ format. Reads were trimmed based on ambiguous bases and quality score and then filtered to eliminate all trimmed reads that were less than 40 bases in length. Approximately 0.3% of reads were removed from each data set.

The RNA-Seq algorithm of CLC Genomics Workbench was used to map the reads from each data set to the BG10 wild type annotated sequence. Note that the BG10 genome does not contain the expression cassettes for the mannosidase and blasticidin resistance gene present in the Man5 and SuperM5 strains.

Gene Expression Profiling. Expression profiles from each of the 4 strains were plotted and clustered. Scatter plots (with R-values) were evaluated for strain to strain comparisons of overall expression profiles. The BG10 and GS115 strains show the tightest correlation (R-value=0.98), followed by the Man5 and SuperM5 strains (R-value=0.95). A slight general upregulation was observed in the OCH1 mutant strains vs. GS115 (R-values of 0.92 and 0.84 for Man5 and SuperM5 respectively). Overall, gene expression patterns are similar amongst the 3 strains (GS115, M5 and SuperM5) when grown on glycerol.

From each of the RNA-Seq data sets mapping to the BG10 strain, the OCH1 mapping was extracted. In the BG10 and GS115 strains, the coverage scale was from 0 to about 75. The expression levels of OCH1 were approximately equal. Sequencing reads were distributed approximately equally across the open reading frame. The expression levels of OCH1 in these two strains were approximately 0.2% that of the most highly expressed genes.

For the SuperM5 strain, the coverage scale was from 0-47. The expression level dropped to approximately half that of the BG10 and GS115 strains. Also, there was no coverage of the N-terminus of the open reading frame. This lack of coverage was the result of the complete deletion of these DNA sequences from the SuperM5 strain.

For the Man5 strain, the coverage scale was from 0-502. There was significantly more coverage of the N-terminal portion of the open reading frame than the C-terminal portion. This disjointed coverage was the result of the duplication of most of the N-terminal portion of the open reading frame in the Man5 strain. The N-terminal portion of the ORF is expressed from DNA upstream of the mannosidase ORF and the mutant form of the C-terminal portion of the ORF was expressed downstream of the mannosidase and blasticidin resistance ORFs. Based on read coverage, the C-terminal portion of the ORF appears to be slightly less abundant in Man5 than in SuperM5.

Mapping of the Man5 and SuperM5 data to the mutant form of the OCH1 ORF shows complete coverage of the mutant OCH1 ORF in both strains, indicating gene expression. The Man₅ strain shows extra coverage in the N-terminal portion of the ORF, for the same reasons described above for the wild type OCH1 ORF mapping.

Mapping of the Man5 and SuperM5 data to the mannosidase ORF shows similar expression levels in the two strains.

Conclusion. Transcriptome analysis has been performed on the GS115, Man₅ and SuperM5 strains. The strains show similar overall gene expression patterns. In the Man and SuperM5 strains, a mutant form of the OCH1 ORF is expressed (polyadenylated mRNA is present). The mannosidaseHDEL ORF is expressed in both strains at approximately the same level.

### Example 7—Trastuzumab Expression in a M5-Blast Like and SuperM5 Strains

In Study 1, the SuperM5 strain described in Example 1 was transformed with an expression vector coding for trastuzumab by electroporation. Zeocin-resistant colonies were screened by the genome PCR using AOX1 primers and Herceptin specific primers. Positive clones of the genome PCR was cultivated and Man5-type trastuzumab expression to the culture supernatants was evaluated by SDS-PAGE.

In Study 2, Pichia strains transformed with an expression vector coding for trastuzumab were screened to select a strain that expressed high levels of trastuzumab. The selected strain was transformed with GlycoSwitch® plasmid (pGlycoSwitch-M5/2 (GAP, BSD), provided by Gent University) by eletroporation. Blasticidin S-resistant colonies were screened by the genome PCR for detecting the pGlycoSwitch-M5 insertion into the OCH1 locus and MDS1 gene presence. Positive clones of the genome PCR was cultivated and Man5-type trastuzumab expression to the culture supernatants was evaluated by SDS-PAGE.

In Study 3, the positive clones obtained in Study 1 (clone 46) and Study 2 (clone 11) were cultivated in a 1 L baffled flask. Trastuzumab expression was induced by replacing with methanol containing medium. 72 hours after methanol induction, trastuzumab was purified using Protein A-affinity resin from the culture supernatants. Productivity of trastuzumab from clone 46 and clone 11 was 3 mg/L and 1.3 mg/L culture, respectively.

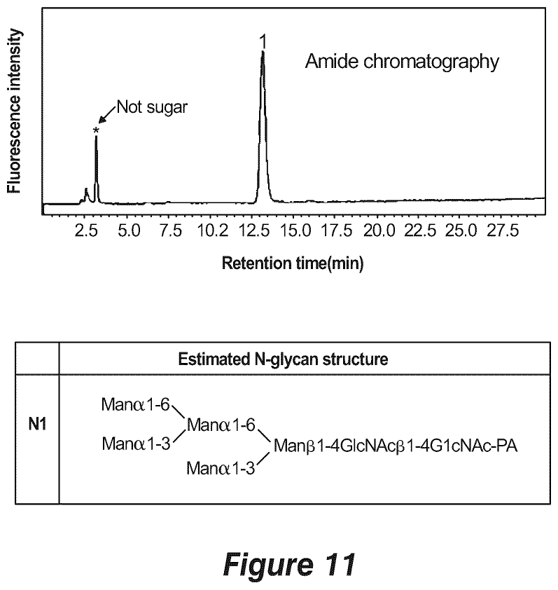

In Study 4, the N-glycan structures of trastuzumab produced in clone 46 (Study 1) and clone 11 (Study 2) were analyzed. The homogeneity of N-glycan structures was assessed in the primary analysis, and the N-glycan structures were identified in the secondary analysis according to searching N-glycan database and HPLC injection along with the standard sample. From these analyses, the N-glycans of trastuzumab obtained from clone 46 (Study 1) were virtually homogeneous and the predominant (or essentially the only) N-glycan was estimated as Man5GlcNac2 from MALDI-TOF mass analysis (FIG. 11). The N-glycan structures of trastuzumab obtained from clone 11 (Study 2) were found to be a mixture of Man5GlcNAc2 to Man8GlcNAc2.

TABLE 7
N-glycan analysis of trastuzumab obtained from Study 2

| | | | | | Quantitative | |
|---|---|---|---|---|---|---|
| | | | | Composition | value | Estimated |
| N-glycan | ODS (GU) | Amide (GU) | MW (Da) | (%) | (pmol/mg) | N-glycan structure |
| N1-1 | 4.7 | 9.7 | 1962 | 7.4 | 161 | (Hexose)₉(HexNAc)₂ |
| N1-2 | | 10.7 | 2124 | 4.6 | 101 | (Hexose)₁₀(HexNAc)₂ |
| N2-1 | 5.0 | 8.8 | 1800 | 22.3 | 487 | Man₈GlcNAc₂ |
| N2-2 | | 10.1 | 2124 | 7.1 | 154 | (Hexose)₁₀(HexNAc)₂ |
| N3 | 5.2 | 7.9 | 1638 | 7.4 | 161 | Man₇GlcNAc₂ |
| N4-1 | 6.1 | 7.0 | 1475 | 16.9 | 370 | Man₆GlcNAc₂ |
| N4-2 | | 7.9 | 1638 | 11.1 | 241 | (Hexose)₇(HexNAc)₂ |
| N5 | 7.3 | 6.0 | 1313 | 22.1 | 481 | Man₅GlcNAc₂ |
| Others | | | | 1.1 | | |
| Total | | | | 100 | | |

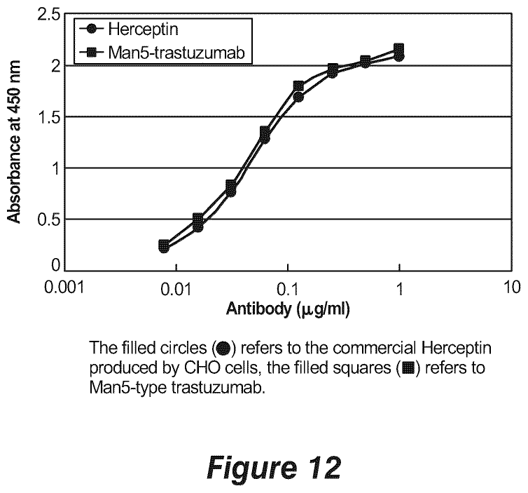

Her2 binding affinity of Man5-type trastuzumab obtained from clone 46 was analyzed in parallel with commercial Herceptin by ELISA and BIAcore assays, was found to have similar HER2-binding activity to the commercial Herceptin. See FIG. 12 and Table 8.

TABLE 8
Kinetic parameters of trastuzumab analyzed on BIAcore

| mAb | kₐ (M⁻¹ s⁻¹) | k_{d} (s⁻¹) | K_{A} (M⁻¹) | K_{D} (nM) |
|---|---|---|---|---|
| Man5-trastuzumab | 2.29 × 10⁵ | 2.43 × 10⁻⁵ | 1.20 × 10¹⁰ | 0.083 |
| CHO Herceptin | 4.25 × 10⁵ | 5.21 × 10⁻⁵ | 8.17 × 10⁹ | 0.12 |
| Pichia trastuzumab | 4.65 × 10⁵ | 8.72 × 10⁻⁵ | 5.33 × 10⁹ | 0.19 |

### Example 8—Analysis of Additional Glycosylated Proteins Expressed in MS-Blast and SuperM5

Genes for Candida antarctica lipases A and B (CalA, 2 N-glycosylation motifs and CalB, 1 N-glycosylation motif) as well as for human serum transferrin (2 N-glycosylation motifs), driven by an AOX1 promoter, were integrated into the genome of the M5-Blast strain as well as the SuperM5 strain, both described in Example 1, via homologous recombination at the AOX1 locus (selection by Zeocin). A plasmid harboring a complementation cassette for histidine auxotrophy next to a synthetic gene coding for native Pichia PDI that is driven by an AOX1 promoter, was co-transformed. Selection was done on solid minimal media with Zeocin.

47 transformants of each combination described above were cultivated and screened for protein abundance and quality with respect to obvious changes in the migration behavior of the secreted proteins on microCE (capillary electrophoresis, GXII, CaliperLS). Mock strain supernatant (GS115) was applied as negative control.

All 3 proteins secreted from the SuperM5 strain showed comparable expression levels as compared to the M5-Blast strain. Furthermore, target protein signals from the SuperM5 supernatants on microCE exhibited a lowered migration time as those from M5-Blast supernatants, shifting to lower apparent molecular weights. It is believed that altered N-glycosylation of secreted proteins from SuperM5 resulted in a lower molecular mass in microscale.

Samples of the supernatants from microscale cultures and those from cultures in a bioreactor were analyzed for its N-glycan compositions. From the samples obtained from microscale culture, 0.5 ml of the medium was diluted with two times the volume of RCM buffer (8 M urea, 3.2 mM EDTA, 360 mM Tris-HCL, PH 8.6). From the bioreactor samples, 0.2 ml medium was used. N-glycans were prepared from these samples following the 96-well on-membrane deglycosylation procedure as published by Laroy et al. (supra). After labeling the dried N-glycans with 8-aminopyrene-1,3,6-trisulphonic acid, the excess of label was removed using size exclusion chromatography (Sephadex G-10 resin). The samples were finally reconstituted in 10 μl of ultrapure water and diluted 10× prior to their injection (80″ at 1.2 kV) in the capillaries (e.l. 36 cm; i.d. 50 m) of an ABI 3730 DNA sequencer. The following settings were applied:
Oven temperature: 60° C. Run voltage: 15 kV
Prerun voltage: 180″ Run time: 1000″
Prerun time: 15 kV

The Genemapper v3.7 was used to analyze the obtained data and structures were assigned to the peaks.

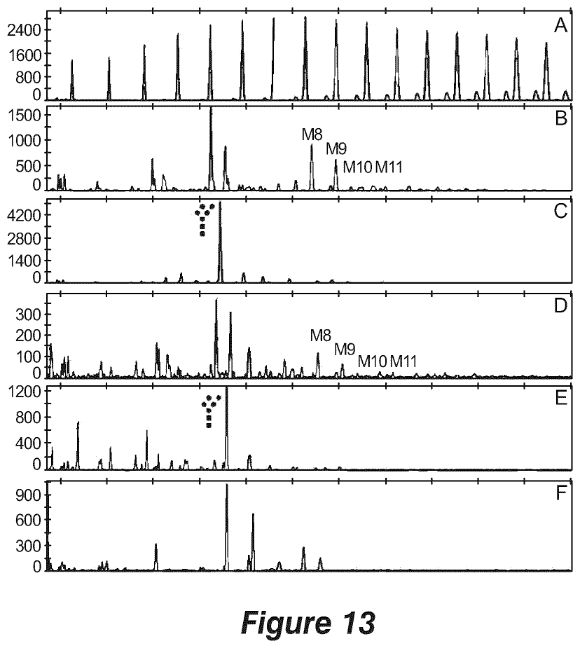

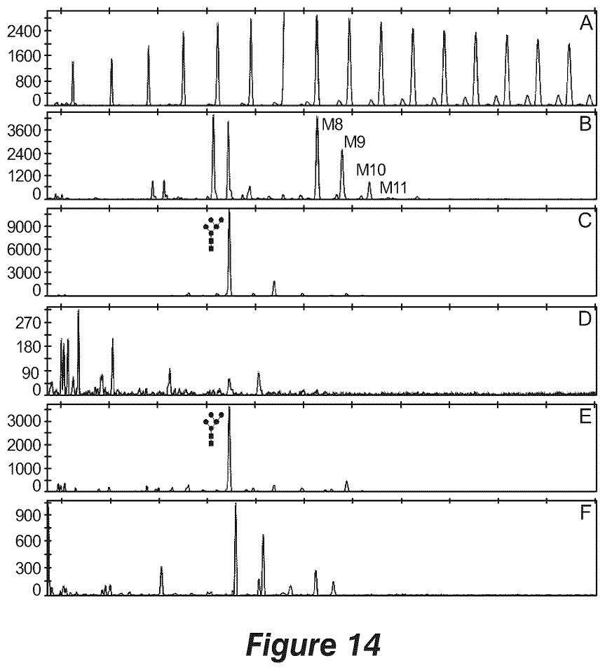

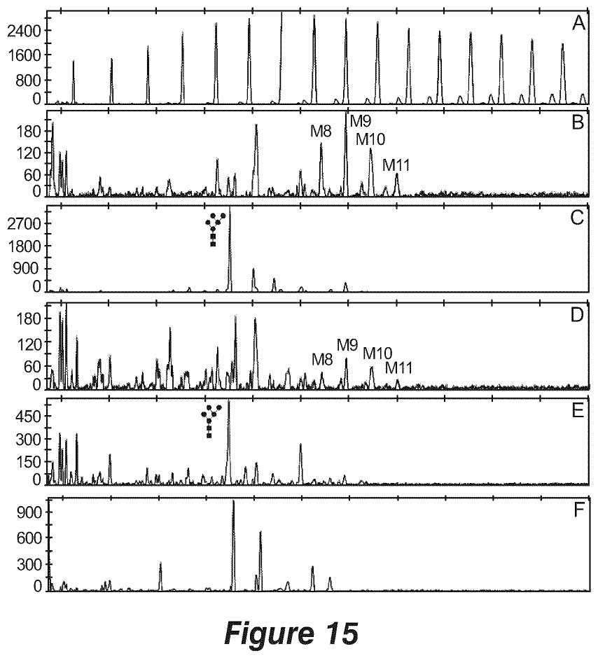

As shown in FIGS. 13-15, the N-glycans of proteins produced from the SuperM5 strain were substantially homogeneous, with Man5GlcNAc2 being the principal N-glycan. In contrast, the N-glycans of proteins produced from the M5-Blast were quite heterogeneous, especially from cultures in a bioreactor.

### Example 9

In this Example, a diploid strain is created by mating the SuperM5 strain described in Example 1 and a wild-type Pichia pastoris strain of a different genetic background. The combination of the two genetic backgrounds allows a determination whether second site repressors or enhancers of the OCH1 disruption phenotype exist in either strain. The diploid is “held together” using two dominant selectable markers in identical genomic locations in each haploid strain. At the diploid OCH1 locus this strain transcribes two different mRNAs; one encoding the wild-type Och1p (from the wild-type haploid genomic copy) and the other encoding the mutant Och1p (from the SuperMan5 haploid genomic copy).

A double-crossover vector containing a Hygromycin B selection marker is constructed that replaces a highly conserved region of Och1p with a V5 epitope tag. This vector is designed so that integration into the diploid genome will, at approximately 50/50 distribution, replace the highly conserved domain in either the wild-type or mutant form of Och1p, creating both epitope insertions in the same starting genetic background. In the case where the vector integrates into the SuperM5 genomic copy of OCH1, the drug selection marker on the vector will be tightly linked to the existing Blasticidin marker adjacent to OCH1. Genomic PCR and DNA sequencing can be used to verify the construction of the two diploid strains, one with the wild-type and one with the mutant form of Och1p epitope tagged.

The diploids are sporulated and random spores grown and analyzed. After growth on non-selective medium, resulting haploid colonies are scored for Hygromycin B resistance. Distribution and growth characteristics of Hygromycin B resistance haploids can determine the lethality or growth deficiency of Och1p inactivation by epitope insertion.

Methods—An existing SuperMan5 strain with a Zeocin resistance marker at the prb1Δ locus is mated with a BG10 haploid strain with a nourseothricin resistance marker at the prb1Δ locus to create the starting diploid strain. The BG10 strain is created from a prb1Δ knockout DNA construct.

A Hygromycin B vector is constructed to replace 14 amino acids in the Och1p sequence (LFARGGLYADMDTML, SEQ ID NO: 92) with the 14 amino acid V5 epitope (GKPIPNPLLGLDST, SEQ ID NO: 93). This retains the full length coding region for both the wild-type and mutant forms of Och1p when integrated into the genome.

The Hygromycin B vector is integrated by homologous recombination into the diploid genome. PCR screening of genomic DNA can be used to verify the chromosome (either SuperMan5 or BG10) and location at which homologous recombination has occurred. PCR products from positive strains are sequenced to verify the replacement of the 14 amino acid domain from Och1p with the V5 tag, making sure the respective ORF lengths are retained in each of the two copies.

The two strains are grown and sporulated, and resultant haploids verified by sensitivity to one or the other drug marker at the prb1Δ locus. Haploids can be visually screened for growth phenotype at the plate level and, if a marked growth distribution is observed, scored for the presence of the V5 tagged construct at either or both of the SuperMan5 or BG10 och1 loci. If all haploids grow equally well, they can be scored for the presence of the Hygromycin B marker. Loss of the Hygromycin B marker on sporulation and subsequent germination will indicate that insertion of the V5 epitope into the Och1p protein is lethal in both the wild-type and mutant cases.

Detection of V5 tagged protein by Western blot in supernatants and extracts of both diploid strains, and, if viable, resultant haploids. As additional experimentation, subcellular location of the wild-type and mutant V5 tagged forms of Och1p can be performed by immunofluorescence on diploid cells.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

The reference numbers recited in the below claims are solely for ease of examination of this patent application, and are exemplary, and are not intended in any way to limit the scope of the claims to the particular features having the corresponding reference numbers in the drawings.

TABLE 1
SEQ ID NO: 1.

| | |
|---|---|
| 1 | AACGTCAAAG ACAGCAATGG AGTCAATATT GATAACACCA CTGGCAGAGC GGTTCGTACG |
| 61 | TCGTTTTGGA GCCGATATGA GGCTCAGCGT GCTAACAGCA CGATTGACAA GAAGACTCTC |
| 121 | GAGTGACAGT AGGTTGAGTA AAGTATTCGC TTAGATTCCC AACCTTCGTT TTATTCTTTC |
| 181 | GTAGACAAAG AAGCTGCATG CGAACATAGG GACAACTTTT ATAAATCCAA TTGTCAAACC |
| 241 | AACGTAAAAC CCTCTGGCAC CATTTTCAAC ATATATTTGT GAAGCAGTAC GCAATATCGA |
| 301 | TAAATACTCA CCGTTGTTTG TAACAGCCCC AACTTGCATA CGCCTTCTAA TGACCTCAAA |
| 361 | TGGATAAGCC GCAGCTTGTG CTAACATACC AGCAGCACCG CCCGCGGTCA GCTGCGCCCA |
| 421 | CACATATAAA GGCAATCTAC GATCATGGGA GGAATTAGTT TTGACCGTCA GGTCTTCAAG |
| 481 | AGTTTTGAAC TCTTCTTCTT GAACTGTGTA ACCTTTTAAA TGACGGGATC TAAATACGTC |
| 541 | ATGGATGAGA TCATGTGTGT AAAAACTGAC TCCAGCATAT GGAATCATTC CAAAGATTGT |
| 601 | AGGAGCGAAC CCACGATAAA AGTTTCCCAA CCTTGCCAAA GTGTCTAATG CTGTGACTTG |
| 661 | AAATCTGGGT TCCTCGTTGA AGACCCTGCG TACTATGCCC AAAAACTTTC CTCCACGAGC |
| 721 | CCTATTAACT TCTCTATGAG TTTCAAATGC CAAACGGACA CGGATTAGGT CCAATGGGTA |
| 781 | AGTGAAAAAC ACAGAGCAAA CCCCAGCTAA TGAGCCGGCC AGTAACCGTC TTGGAGCTGT |
| 841 | TTCATAAGAG TCATTAGGGA TCAATAACGT TCTAATCTGT TCATAACATA CAAATTTTAT |
| 901 | GGCTGCATAG GGAAAAATTC TCAACAGGGT AGCCGAATGA CCCTGATATA GACCTGCGAC |
| 961 | ACCATCATAC CCATAGATCT GCCTGACAGC CTTAAAGAGC CCGCTAAAAG ACCCGGAAAA |
| 1021 | CCGAGAGAAC TCTGGATTAG CAGTCTGAAA AAGAATCTTC ACTCTGTCTA GTGGAGCAAT |
| 1081 | TAATGTCTTA GCGGCACTTC CTGCTACTCC GCCAGCTACT CCTGAATAGA TCACATACTG |
| 1141 | CAAAGACTGC TTGTCGATGA CCTTGGGGTT ATTTAGCTTC AAGGGCAATT TTTGGGACAT |
| 1201 | TTTGGACACA GGAGACTCAG AAACAGACAC AGAGCGTTCT GAGTCCTGGT GCTCCTGACG |
| 1261 | TAGGCCTAGA ACAGGAATTA TTGGCTTTAT TTGTTTGTCC ATTTCATAGG CTTGGGGTAA |
| 1321 | TAGATAGATG ACAGAGAAAT AGAGAAGACC TAATATTTTT TGTTCATGGC AAATCGCGGG |
| 1381 | TTCGCGGTCG GGTCACACAC GGAGAAGTAA TGAGAAGAGC TGGTAATCTG GGGTAAAAGG |
| 1441 | GTTCAAAAGA AGGTCGCCTG GTAGGGATGC AATACAAGGT TGTCTTGGAG TTTACATTGA |
| 1501 | CCAGATGATT TGGCTTTTTC TCTGTTCAAT TCACATTTTT CAGCGAGAAT CGGATTGACG |
| 1561 | GAGAAATGGC GGGGTGTGGG GTGGATAGAT GGCAGAAATG CTCGCAATCA CCGCGAAAGA |
| 1621 | AAGACTTTAT GGAATAGAAC TACTGGGTGG TGTAAGGATT ACATAGCTAG TCCAATGGAG |
| 1681 | TCCGTTGGAA AGGTAAGAAG AAGCTAAAAC CGGCTAAGTA ACTAGGGAAG AATGATCAGA |
| 1741 | CTTTGATTTG ATGAGGTCTG AAAATACTCT GCTGCTTTTT CAGTTGCTTT TTCCCTGCAA |
| 1801 | CCTATCATTT TCCTTTTCAT AAGCCTGCCT TTTCTGTTTT CACTTATATG AGTTCCGCCG |
| 1861 | AGACTTCCCC AAATTCTCTC CTGGAACATT CTCTATCGCT CTCCTTCCAA GTTGCGCCCC |
| 1921 | CTGGCACTGC CTAGTAATAT TACCACGCGA CTTATATTCA GTTCCACAAT TTCCAGTGTT |
| 1981 | CGTAGCAAAT ATCATCAGCC TACCGTTCGT ATAGCATACA TTATACGAAC GGTACTTTTT |
| 2041 | TGTAGAAATG TCTTGGTGTC CTCGTCCAAT CAGGTAGCCA TCTCTGAAAT ATCTGGCTCC |
| 2101 | GTTGCAACTC CGAACGACCT GCTGGCAACG TAAAATTCTC CGGGGTAAAA CTTAAATGTG |
| 2161 | GAGTAATGGA ACCAGAAACG TCTCTTCCCT TCTCTCTCCT TCCACCGCCC GTTACCGTCC |
| 2221 | CTAGGAAATT TTACTCTGCT GGAGAGCTTC TTCTACGGCC CCCTTGCAGC AATGCTCTTC |
| 2281 | CCAGCATTAC GTTGCGGGTA AAACGGAGGT CGTGTACCCG ACCTAGCAGC CCAGGGATGG |
| 2341 | AAAAGTCCCG GCCGTCGCTG GCAATAATAG CGGGCGGACG CATGTCATGA GATTATTGGA |
| 2401 | AACCACCAGA ATCGAATATA AAAGGCGAAC ACCTTTCCCA ATTTTGGTTT CTCCTGACCC |
| 2461 | AAAGACTTTA AATTTAATTT ATTTGTCCCT ATTTCAATCA ATTGAACAAC TATTTCGCGA |
| 2521 | AACGATGAGA TTTCCTTCAA TTTTTACTGC TGTTTTATTC GCAGCATCCT CCGCATTAGC |
| 2581 | TGCTCCAGTC AACACTACAA CAGAAGATGA AACGGCACAA ATTCCGGCTG AAGCTGTCAT |
| 2641 | CGGTTACTCA GATTTAGAAG GGGATTTCGA TGTTGCTGTT TTGCCATTTT CCAACAGCAC |
| 2701 | AAATAACGGG TTATTGTTTA TAAATACTAC TATTGCCAGC ATTGCTGCTA AAGAAGAAGG |
| 2761 | GGTATCTCTC GAGAAAAGAG AGGCTGAAGC TGAATTCGCC ACAAAACGTG GATCTCCCAA |
| 2821 | CCCTACGAGG GCGGCAGCAG TCAAGGCCGC ATTCCAGACG TCGTGGAACG CTTACCACCA |
| 2881 | TTTTGCCTTT CCCCATGACG ACCTCCACCC GGTCAGCAAC AGCTTTGATG ATGAGAGAAA |
| 2941 | CGGCTGGGGC TCGTCGGCAA TCGATGGCTT GGACACGGCT ATCCTCATGG GGGATGCCGA |
| 3001 | CATTGTGAAC ACGATCCTTC AGTATGTACC GCAGATCAAC TTCACCACGA CTGCGGTTGC |
| 3061 | CAACCAAGGC ATCTCCGTGT TCGAGACCAA CATTCGGTAC CTCGGTGGCC TGCTTTCTGC |
| 3121 | CTATGACCTG TTGCGAGGTC CTTTCAGCTC CTTGGCGACA AACCAGACCC TGGTAAACAG |
| 3181 | CCTTCTGAGG CAGGCTCAAA CACTGGCCAA CGGCCTCAAG GTTGCGTTCA CCACTCCCAG |
| 3241 | CGGTGTCCCG GACCCTACCG TCTTCTTCAA CCCTACTGTC CGGAGAAGTG GTGCATCTAG |
| 3301 | CAACAACGTC GCTGAAATTG GAAGCCTGGT GCTCGAGTGG ACACGGTTGA GCGACCTGAC |
| 3361 | GGGAAACCCG CAGTATGCCC AGCTTGCGCA GAAGGGCGAG TCGTATCTCC TGAATCCAAA |
| 3421 | GGGAAGCCCG GAGGCATGGC CTGGCCTGAT TGGAACGTTT GTCAGCACGA GCAACGGTAC |
| 3481 | CTTTCAGGAT AGCAGCGGCA GCTGGTCCGG CCTCATGGAC AGCTTCTACG AGTACCTGAT |
| 3541 | CAAGATGTAC CTGTACGACC CGGTTGCGTT TGCACACTAC AAGGATCGCT GGGTCCTTGC |
| 3601 | TGCCGACTCG ACCATTGCGC ATCTCGCCTC TCACCCGTCG ACGCGCAAGG ACTTGACCTT |
| 3661 | TTTGTCTTCG TACAACGGAC AGTCTACGTC GCCAAACTCA GGACATTTGG CCAGTTTTGC |
| 3721 | CGGTGGCAAC TTCATCTTGG GAGGCATTCT CCTGAACGAG CAAAAGTACA TTGACTTTGG |
| 3781 | AATCAAGCTT GCCAGCTCGT ACTTTGCCAC GTACAACCAG ACGGCTTCTG GAATCGGCCC |
| 3841 | CGAAGGCTTC GCGTGGGTGG ACAGCGTGAC GGGCGCCGGC GGCTCGCCGC CCTCGTCCCA |
| 3901 | GTCCGGGTTC TACTCGTCGG CAGGATTCTG GGTGACGGCA CCGTATTACA TCCTGCGGCC |
| 3961 | GGAGACGCTG GAGAGCTTGT ACTACGCATA CCGCGTCACG GGCGACTCCA AGTGGCAGGA |
| 4021 | CCTGGCGTGG GAAGCGTTCA GTGCCATTGA GGACGCATGC CGCGCCGGCA GCGCGTACTC |
| 4081 | GTCCATCAAC GACGTGACGC AGGCCAACGG CGGGGGTGCC TCTGACGATA TGGAGAGCTT |
| 4141 | CTGGTTTGCC GAGGCGCTCA AGTATGCGTA CCTGATCTTT GCGGAGGAGT CGGATGTGCA |
| 4201 | GGTGCAGGCC AACGGCGGGA ACAAATTTGT CTTTAACACG GAGGCGCACC CCTTTAGCAT |
| 4261 | CCGTTCATCA TCACGACGGG GCGGCCACCT TGCTCACGAC GAGTTGTAAT CTAGGGCGGC |
| 4321 | CGCCAGCTTG GGCCCGAACA AAAACTCATC TCAGAAGAGG ATCTGAATAG CGCCGTCGAC |
| 4381 | CATCATCATC ATCATCATTG AGTTTTAGCC TTAGACATGA CTGTTCCTCA GTTCAAGTTG |
| 4441 | GGCACTTACG AGAAGACCGG TCTTGCTAGA TTCTAATCAA GAGGATGTCA GAATGCCATT |
| 4501 | TGCCTGAGAG ATGCAGGCTT CATTTTTGAT ACTTTTTTAT TTGTAACCTA TATAGTATAG |
| 4561 | GATTTTTTTT GTCATTTTGT TTCTTCTCGT ACGAGCTTGC TCCTGATCAG CCTATCTCGC |
| 4621 | AGCTGATGAA TATCTTGTGG TAGGGGTTTG GGAAAATCAT TCGAGTTTGA TGTTTTTCTT |
| 4681 | GGTATTTCCC ACTCCTCTTC AGAGTACAGA AGATTAAGTG AGACCTTCGT TTGTGCGGAT |
| 4741 | CCCCCACACA CCATAGCTTC AAAATGTTTC TACTCCTTTT TTACTCTTCC AGATTTTCTC |
| 4801 | GGACTCCGCG CATCGCCGTA CCACTTCAAA ACACCCAAGC ACAGCATACT AAATTTCCCC |
| 4861 | TCTTTCTTCC TCTAGGGTGT CGTTAATTAC CCGTACTAAA GGTTTGGAAA AGAAAAAAGA |
| 4921 | GACCGCCTCG TTTCTTTTTC TTCGTCGAAA AAGGCAATAA AAATTTTTAT CACGTTTCTT |
| 4981 | TTTCTTGAAA ATTTTTTTTT TTGATTTTTT TCTCTTTCGA TGACCTCCCA TTGATATTTA |
| 5041 | AGTTAATAAA CGGTCTTCAA TTTCTCAAGT TTCAGTTTCA TTTTTCTTGT TCTATTACAA |
| 5101 | CTTTTTTTAC TTCTTGCTCA TTAGAAAGAA AGCATAGCAA TCTAATCTAA GGGCGGTGTT |
| 5161 | GACAATTAAT CATCGGCATA GTATATCGGC ATAGTATAAT ACGACAAGGT GAGGAACTAA |
| 5221 | ACCATGGCCA AGCCTTTGTC TCAAGAAGAA TCCACCCTCA TTGAAAGAGC AACGGCTACA |
| 5281 | ATCAACAGCA TCCCCATCTC TGAAGACTAC AGCGTCGCCA GCGCAGCTCT CTCTAGCGAC |
| 5341 | GGCCGCATCT TCACTGGTGT CAATGTATAT CATTTTACTG GGGGACCTTG TGCAGAACTC |
| 5401 | GTGGTGCTGG GCACTGCTGC TGCTGCGGCA GCTGGCAACC TGACTTGTAT CGTCGCGATC |
| 5461 | GGAAATGAGA ACAGGGGCAT CTTGAGCCCC TGCGGACGGT GCCGACAGGT GCTTCTCGAT |
| 5521 | CTGCATCCTG GGATCAAAGC CATAGTGAAG GACAGTGATG GACAGCCGAC GGCAGTTGGG |
| 5581 | ATTCGTGAAT TGCTGCCCTC TGGTTATGTG TGGGAGGGCT AAGCACTTCG TGGCCGAGGA |
| 5641 | GCAGGACTGA CACGTCCGAC GCGGCCCGAC GGGTCCGAGG CCTCGGAGAT CCGTCCCCCT |
| 5701 | TTTCCTTTGT CGATATCATG TAATTAGTTA TGTCACGCTT ACATTCACGC CCTCCCCCCA |
| 5761 | CATCCGCTCT AACCGAAAAG GAAGGAGTTA GACAACCTGA AGTCTAGGTC CCTATTTATT |
| 5821 | TTTTTATAGT TATGTTAGTA TTAAGAACGT TATTTATATT TCAAATTTTT CTTTTTTTTC |
| 5881 | TGTACAGACG CGTGTACGCA TGTAACATTA TACTGAAAAC CTTGCTTGAG AAGGTTTTGG |
| 5941 | GACGCTCGAA GGCTTTAATT TGCAAGCTGG AGACCAACAT GTGAGCAAAA GGCCAGCAAA |
| 6001 | AGGCCAGGAA CCGTAAAAAG GCCGCGTTGC TGGCGTTTTT CCATAGGCTC CGCCCCCCTG |
| 6061 | ACGAGCATCA CAAAAATCGA CGCTCAAGTC AGAGGTGGCG AAACCCGACA GGACTATAAA |
| 6121 | GATACCAGGC GTTTCCCCCT GGAAGCTCCC TCGTGCGCTC TCCTGTTCCG ACCCTGCCGC |
| 6181 | TTACCGGATA CCTGTCCGCC TTTCTCCCTT CGGGAAGCGT GGCGCTTTCT CATAGCTCAC |
| 6241 | GCTGTAGGTA TCTCAGTTCG GTGTAGGTCG TTCGCTCCAA GCTGGGCTGT GTGCACGAAC |
| 6301 | CCCCCGTTCA GCCCGACCGC TGCGCCTTAT CCGGTAACTA TCGTCTTGAG TCCAACCCGG |
| 6361 | TAAGACACGA CTTATCGCCA CTGGCAGCAG CCACTGGTAA CAGGATTAGC AGAGCGAGGT |
| 6421 | ATGTAGGCGG TGCTACAGAG TTCTTGAAGT GGTGGCCTAA CTACGGCTAC ACTAGAAGAA |
| 6481 | CAGTATTTGG TATCTGCGCT CTGCTGAAGC CAGTTACCTT CGGAAAAAGA GTTGGTAGCT |
| 6541 | CTTGATCCGG CAAACAAACC ACCGCTGGTA GCGGTGGTTT TTTTGTTTGC AAGCAGCAGA |
| 6601 | TTACGCGCAG AAAAAAAGGA TCTCAAGAAG ATCCTTTGAT CTTTTCTACG GGGTCTGACG |
| 6661 | CTCAGTGGAA CGAAAACTCA CGTTAAGGGA TTTTGGTCAT GAGATCAGAT CTAACATCCA |
| 6721 | TAATCGTATT CGCCGTTTCT GTCATTTGCG TTTTGTACGG ACCCTCACAA CAATTATCAT |
| 6781 | CTCCAAAAAT AGACTATGAT CCATTGACGC TCCGATCACT TGATTTGAAG ACTTTGGAAG |
| 6841 | CTCCTTCACA GTTGAGTCCA GGCACCGTAG AAGATAATCT TCGAAGACAA TTGGAGTTTC |
| 6901 | ATTTTCCTTA CCGCAGTTAC GAACCTTTTC CCCAACATAT TTGGCAAACG TGGAAAGTTT |
| 6961 | CTCCCTCTGA TAGTTCCTTT CCGAAAAACT TCAAAGACTT AGGTGAAAGT TGGCTGCAAA |
| 7021 | GGTCCCCAAA TTATGATCAT TTTGTGATAC CCGATGATGC AGCATGGGAA CTTATTCACC |
| 7081 | ATGAATACGA ACGTGTACCA GAAGTCTTGG AAGCTTTCCA CCTGCTACCA GAGCCCATTC |
| 7141 | TAAAGGCCGA TTTTTTCAGG TATTTGATTC TTTTTGCCCG TGGAGGACTG TATGCTGACA |
| 7201 | TGGACACTAT GTTATTAAAA CCAATAGAAT CGTGGCTGAC TTTCAATGAA ACTATTGGTG |
| 7261 | GAGTAAAAAA CAATGCTGGG TTGGTCATTG GTATTGAGGC TGATCCTGAT AGACCTGATT |
| 7321 | GGCACGACTG GTATGCTAGA AGGATACAAT TTTGCCAATG GGCAATTCAG TCCAAACGAG |
| 7381 | GACACCCAGC ACTGCGTGAA CTGATTGTAA GAGTTGTCAG CACGACTTTA CGGAAAGAGA |
| 7441 | AAAGCGGTTA CTTGAACATG GTGGAAGGAA AGGATCGTGG AAGTGATGTG ATGGACTGGA |
| 7501 | CGGGTCCAGG AATATTTACA GACACTCTAT TTGATTATAT GACTAATGTC AATACAACAG |
| 7561 | GCCACTCAGG CCAAGGAATT GGAGCTGGCT CAGCGTATTA CAATGCCTTA TCGTTGGAAG |
| 7621 | AACGTGATGC CCTCTCTGCC CGCCCGAACG GAGAGATGTT AAAAGAGAAA GTCCCAGGTA |
| 7681 | AATATGCACA GCAGGTTGTT TTATGGGAAC AATTTACCAA CCTGCGCTCC CCCAAATTAA |
| 7741 | TCGACGATAT TCTTATTCTT CCGATCACCA GCTTCAGTCC AGGGATTGGC CACAGTGGAG |
| 7801 | CTGGAGATTT GAACCATCAC CTTGCATATA TTAGGCATAC ATTTGAAGGA AGTTGGAAGG |
| 7861 | ACTAAAGAAA GCTAGAGTAA AATAGATATA GCGAGATTAG AGAATGAATA CCTTCTTCTA |
| 7921 | AGCGATCGTC CGTCATCATA GAATATCATG GACTGTATAG TTTTTTTTTT GTACATATAA |
| 7981 | TGATTAAACG GTCATCCAAC ATCTCGTTGA CAGATCTCTC AGTACGCGAA ATCCCTGACT |
| 8041 | ATCAAAGCAA GAACCGATGA AGAAAAAAAC AACAGTAACC CAAACACCAC AACAAACACT |
| 8101 | TTATCTTCTC CCCCCCAACA CCAATCATCA AAGAGATGTC GGAACCAAAC ACCAAGAAGC |
| 8161 | AAAAACTAAC CCCATATAAA AACATCCTGG TAGATAATGC TGGTAACCCG CTCTCCTTCC |
| 8221 | ATATTCTGGG CTACTTCACG AAGTCTGACC GGTCTCAGTT GATCAACATG ATCCTCGAAA |
| 8281 | TGGGTGGCAA GATCGTTCCA GACCTGCCTC CTCTGGTAGA TGGAGTGTTG TTTTTGACAG |
| 8341 | GGGATTACAA GTCTATTGAT GAAGATACCC TAAAGCAACT GGGGGACGTT CCAATATACA |
| 8401 | GAGACTCCTT CATCTACCAG TGTTTTGTGC ACAAGACATC TCTTCCCATT GACACTTTCC |
| 8461 | GAATTGACAA GAACGTCGAC TTGGCTCAAG ATTTGATCAA TAGGGCCCTT CAAGAGTCTG |
| 8521 | TGGATCATGT CACTTCTGCC AGCACAGCTG CAGCTGCTGC TGTTGTTGTC GCTACCAACG |
| 8581 | GCCTGTCTTC TAAACCAGAC GCTCGTACTA GCAAAATACA GTTCACTCCC GAAGAAGATC |
| 8641 | GTTTTATTCT TGACTTTGTT AGGAGAAATC CTAAACGAAG AAACACACAT CAACTGTACA |
| 8701 | CTGAGCTCGC TCAGCACATG AAAAACCATA CGAATCATTC TATCCGCCAC AGATTTCGTC |
| 8761 | GTAATCTTTC CGCTCAACTT GATTGGGTTT ATGATATCGA TCCATTGACC AACCAACCTC |
| 8821 | GAAAAGATGA AAACGGGAAC TACATCAAGG TACAAGATCT TCCACAAGGA ATTCGTGGTC |
| 8881 | ATTATTCTGC CCAAGATGAT TACAATTTGT GTTTATCGGT TCAACCTTTC ATTGAATCTG |
| 8941 | TAGATGAGAC AACAGGCCAA GAATTTTTCA AACCTCTGAA AGGTGTATTT GATGACTTGG |
| 9001 | AATCTCGCTT TCCTCACCAT ACAAAGACTT CCTGGAGAGA CAGATTCAGA AAGTTTGCCT |
| 9061 | CTAAATACGG TGTTCGTCAG TACATCGCGT ATTATGAAAA GACTGTTGAA CTCAATGGTG |
| 9121 | TTCCTAATCC GATGACGAAC TTTACCTCAA AGGCTTCCAT TGAAAAATTT AGAGAAAGAC |
| 9181 | GCGGGACTTC ACGTAACAGT GGCCTTCCAG GCCCGGTTGG TGTAGAAGCT GTAAGCTCTT |
| 9241 | TGGACCACAT ATCCCCATTG GTCACATCTA ATTCCAATTC TGCAGCTGCT GCAGCTGCTG |
| 9301 | CCGCAGCAGT TGCAGCCTCT GCCTCTGCTT CTTCAGCTCC TAATACTTCA ACTACCAATT |
| 9361 | TCTTTGAACA GGAGAATATT GCCCAAGTTC TCTCTGCACA TAACAACGAG CAGTCTATTG |
| 9421 | CAGAAGTTAT TGAGTCCGCA CAGAATGTCA ACACCCATGA AAGTGAACCT ATAGCTGATC |
| 9481 | ATGTTCGAAA AAATCTTACA GACGATGAAT TGCTTGACAA AATGGATGAT ATTTTAAGCT |
| 9541 | CCAGAAGTCT AGGCGGACTA GATGACTTGA TAAAGATCCT CTACACTGAG CTGGGATTTG |
| 9601 | CTCATCGTTA TACCGAATTT CTTTTTACCT CATGTTCTGG TGATGTGATT TTCTTCCGAC |
| 9661 | CATTAGTGGA ACATTTCCTT CTTACTGGTG AGTGGGAGCT GGAGAATACT CGTGGCATCT |
| 9721 | GGACCGGTCG TCAAGACGAA ATGCTACGTG CTAGCAATCT AGATGACCTG CACAAGTTAA |
| 9781 | TTGACCTGCA TGGGAAAGAA CGTGTTGAGA CCAGAAGAAA AGCCATCAAG GGAGAATGAT |
| 9841 | CATAAGAAAT GAAAAACGTA TAAGT |

TABLE 2
SEQ ID NO: 2.

(M)AKADGSLLY YNPHNPPRRY YFYMAIFAVS VICVLYGPSQ
QLSSPKIDYD PLTLRSLDLK TLEAPSQLSP GTVEDNLRRQ
LEFHFPYRSY EPFPQHIWQT WKVSPSDSSF PKNFKDLGES
WLQRSPNYDH FVIPDDAAWE LIHHEYERVP EVLEAFHLLP
EPILKADFFR YLILFARGGL YADMDTMLLK PIESWLTFNE
TIGGVKNNAG LVIGIEADPD RPDWHDWYAR RIQFCQWAIQ
SKRGHPALRE LIVRVVSTTL RKEKSGYLNM VEGKDRGSDV
MDWTGPGIFT DTLFDYMTNV NTTGHSGQGI GAGSAYYNAL
SLEERDALSA RPNGEMLKEK VPGKYAQQVV LWEQFTNLRS
PKLIDDILIL PITSFSPGIG HSGAGDLNHH LAYIRHTFEG
SWKD

TABLE 3

| | Amino acids | |
|---|---|---|
| Nucleotides | corresponding | |
| deleted from | to deleted | |
| Upstream OCH1 | nucleotides | Description |
| GCG AAG GCA GAT | AKADG | 5 AAs deleted |
| GGC (SEQ ID NO: 29) | (SEQ ID NO: 4) | from Upstream |
| | | OCH1 portion |
| GCG AAG GCA GAT | AKADGS | 6 AAs deleted |
| GGC AGT | (SEQ ID NO: 5) | from Upstream |
| (SEQ ID NO: 30) | | OCH1 portion |
| GCG AAG GCA GAT | AKADGSL | 7 AAs deleted |
| GGC AGT TTG | (SEQ ID NO: 6) | from Upstream |
| (SEQ ID NO: 31) | | OCH1 portion |
| GCG AAG GCA GAT | AKADGSLL | 8 AAs deleted |
| GGC AGT TTG CTC | (SEQ ID NO: 7) | from Upstream |
| (SEQ ID NO: 32) | | OCH1 portion |
| GCG AAG GCA GAT | AKADGSLLY | 9 AAs deleted |
| GGC AGT TTG CTC | (SEQ ID NO: 8) | from Upstream |
| TAC (SEQ ID NO: 33) | | OCH1 portion |
| GCG AAG GCA GAT | AKADGSLLYY | 10 AAs deleted |
| GGC AGT TTG CTC | (SEQ ID NO: 9) | from Upstream |
| TAC TAT (SEQ ID | | OCH1 portion |
| NO: 34) | | |
| ATG GCG AAG GCA | MAKADG | 6 AAs deleted |
| GAT GGC | (SEQ ID NO: 10) | from Upstream |
| (SEQ ID NO: 35) | | OCH1 portion |
| ATG GCG AAG GCA | MAKADGS | 7 AAs deleted |
| GAT GGC AGT | (SEQ ID NO: 11) | from Upstream |
| (SEQ ID NO: 36) | | OCH1 portion |
| ATG GCG AAG GCA | MAKADGSL | 8 AAs deleted |
| GAT GGC AGT TTG | (SEQ ID NO: 12) | from Upstream |
| (SEQ ID NO: 37) | | OCH1 portion |
| ATG GCG AAG GCA | MAKADGSLL | 9 AAs deleted |
| GAT GGC AGT TTG | (SEQ ID NO: 13) | from Upstream |
| CTC (SEQ ID NO: 38) | | OCH1 portion |
| ATG GCG AAG GCA | MAKADGSLLY | 10 AAs deleted |
| GAT GGC AGTTTG | (SEQ ID NO: 14) | from Upstream |
| CTC TAC (SEQ ID | | OCH1 portion |
| NO: 39) | | |
| ATG GCG AAG GCA | MAKADGSLLYY | 11 AAs deleted |
| GAT GGC AGT TTG | (SEQ ID NO: 15) | from Upstream |
| CTC TAC TAT | | OCH1 portion |
| (SEQ ID NO: 40) | | |
| ATG GCG AAG GCA | MAKADGSLLYYN | 12 AAs deleted |
| GAT GGC AGT TTG | (SEQ ID NO: 16) | from Upstream |
| CTC TAC TAT AAT | | OCH1 portion |
| (SEQ ID NO: 41) | | |
| ATG GCG AAG GCA | MAKADGSLLYYNP | 13 AAs deleted |
| GAT GGC AGT TTG | (SEQ ID NO: 17) | from Upstream |
| CTC TAC TAT AAT | | OCH1 portion |
| CCT (SEQ ID NO: 42) | | |
| ATG GCG AAG GCA | MAKADGSLLYYNPH | 14 AAs deleted |
| GAT GGC AGT TTG | (SEQ ID NO: 18) | from Upstream |
| CTC TAC TAT AAT | | OCH1 portion |
| CCT CAC | | |
| (SEQ ID NO: 43) | | |
| ATG GCG AAG GCA | MAKADGSLLYYNPHN | 15 AAs deleted |
| GAT GGC AGT TTG | (SEQ ID NO: 19) | from Upstream |
| CTC TAC TAT AAT | | OCH1 portion |
| CCT CAC AAT | | |
| (SEQ ID NO: 44) | | |
| ATG GCG AAG GCA | MAKADGSLLYYNPHNP | 16 AAs deleted |
| GAT GGC AGT TTG | (SEQ ID NO: 20) | from Upstream |
| CTC TAC TAT AAT | | OCH1 portion |
| CCT CAC AAT CCA | | |
| (SEQ ID NO: 45) | | |
| ATG GCG AAG GCA | MAKADGSLLYYNPHNP | 17 AAs deleted |
| GAT GGC AGT TTG | P | from Upstream |
| CTC TAC TAT AAT | (SEQ ID NO: 21) | OCH1 portion |
| CCT CAC AAT CCA | | |
| CCC (SEQ ID NO: 46) | | |
| ATG GCG AAG GCA | MAKADGSLLYYNPHNP | 18 AAs deleted |
| GAT GGC AGT TTG | PR | from Upstream |
| CTC TAC TAT AAT | (SEQ ID NO: 22) | OCH1 portion |
| CCT CAC AAT CCA | | |
| CCC AGA (SEQ ID | | |
| NO: 47) | | |
| ATG GCG AAG GCA | MAKADGSLLYYNPHNP | 19 AAs deleted |
| GAT GGC AGT TTG | PRR | from Upstream |
| CTC TAC TAT AAT | (SEQ ID NO: 23) | OCH1 portion |
| CCT CAC AAT CCA | | |
| CCC AGA AGG | | |
| (SEQ ID NO: 48) | | |
| ATG GCG AAG GCA | MAKADGSLLYYNPHNP | 20 AAs deleted |
| GAT GGC AGT TTG | PRRY | from Upstream |
| CTC TAC TAT AAT | (SEQ ID NO: 24) | OCH1 portion |
| CCT CAC AAT CCA | | |
| CCC AGA AGG TAT | | |
| (SEQ ID NO: 49) | | |
| ATG GCG AAG GCA | MAKADGSLLYYNPHNP | 21 AAs deleted |
| GAT GGC AGT TTG | PRRYY | from Upstream |
| CTC TAC TAT AAT | (SEQ ID NO: 25) | OCH1 portion |
| CCT CAC AAT CCA | | |
| CCC AGA AGG TAT | | |
| TAC (SEQ ID NO: 50) | | |
| GCG AAG GCA GAT | AKADGSLLYYNPHNPP | 24 AAs deleted |
| GGC AGT TTG CTC | RRYYFYMA | from Upstream |
| TAC TAT AAT CCT | (SEQ ID NO: 26) | OCH1 portion |
| CACA ATC CAC CCA | | |
| GAA GGT ATT ACT | | |
| TCT ACA TGG CTA | | |
| (SEQ ID NO: 51) | | |
| ATG GCG AAG GCA | MAKADGSLLYYNPHNP | 25 AAs deleted |
| GAT GGC AGT TTG | PRRYYFYMA | from Upstream |
| CTC TAC TAT AAT | (SEQ ID NO: 27) | OCH1 portion |
| CCT CACA ATC CAC | | |
| CCA GAA GGT ATT | | |
| ACT TCT ACA TGG | | |
| CTA (sSEQ ID NO: 52) | | |

TABLE 4
SEQ ID NO: 53.

| | |
|---|---|
| 1 | AACGTCAAAG ACAGCAATGG AGTCAATATT GATAACACCA CTGGCAGAGC GGTTCGTACG |
| 61 | TCGTTTTGGA GCCGATATGA GGCTCAGCGT GCTAACAGCA CGATTGACAA GAAGACTCTC |
| 121 | GAGTGACAGT AGGTTGAGTA AAGTATTCGC TTAGATTCCC AACCTTCGTT TTATTCTTTC |
| 181 | GTAGACAAAC AAGCTGCATG CGAACATAGG GACAACTTTT ATAAATCCAA TTGTCAAACC |
| 241 | AACGTAAAAC CCTCTGGCAC CATTTTCAAC ATATATTTGT GAAGCAGTAC GCAATATCGA |
| 301 | TAAATACTGA GCGTTGTTTG TAACAGCCCC AACTTGCATA CGCCTTCTAA TGACCTCAAA |
| 361 | TGGATAAGCC GCAGCTTGTG CTAACATACC AGCAGCACCG CCCGCGGTCA GCTGCGCCCA |
| 421 | CACATATAAA GGCAATCTAC GATCATGGGA GGAATTAGTT TTGACCGTCA GGTCTTCAAG |
| 481 | AGTTTTGAAC TCTTCTTCTT GAACTGTGTA ACCTTTTAAA TGACGGGATC TAAATACGTC |
| 541 | ATGGATGAGA TCATGTGTGT AAAAACTGAC TCCAGCATAT GGAATCATTC CAAAGATTGT |
| 601 | AGGAGCGAAC CCACGATAAA AGTTTCCCAA CCTTGCCAAA GTGTCTAATG CTGTGACTTG |
| 661 | AAATCTGGGT TCCTCGTTGA AGACCCTGCG TACTATGCCG AAAAACTTTC CTCCACGAGC |
| 721 | CCTATTAACT TCTCTATGAG TTTCAAATGC CAAACGGACA CGGATTAGGT CCAATGGGTA |
| 781 | AGTGAAAAAC ACAGAGCAAA CCCCAGCTAA TGAGCCGGCC AGTAACCGTC TTGGAGCTGT |
| 841 | TTCATAAGAC TCATTAGGGA TCAATAACGT TCTAATCTGT TCATAACATA CAAATTTTAT |
| 901 | GGCTGCATAG GGAAAAATTC TCAACAGGGT AGCCGAATGA CCCTGATATA GACCTGCGAC |
| 961 | ACCATCATAC CCATAGATCT GCCTGACAGC CTTAAAGAGC CCGCTAAAAG ACCCGGAAAA |
| 1021 | CCGAGAGAAC TCTGGATTAG CAGTCTGAAA AAGAATCTTC ACTCTGTCTA GTGGAGCAAT |
| 1081 | TAATGTCTTA GCGGCACTTC CTGCTACTCC GCCAGCTACT CCTGAATAGA TCACATACTG |
| 1141 | CAAAGACTGC TTGTCGATGA CCTTGGGGTT ATTTAGCTTC AAGGCCAATT TTTGGGACAT |
| 1201 | TTTGGACACA GGAGACTCAG AAACAGACAC AGAGCGTTCT GAGTCCTGGT GCTCCTGACG |
| 1261 | TAGGCCTAGA ACAGCAATTA TTGGCTTTAT TTGTTTGTCC ATTTCATAGG CTTGGGGTAA |
| 1321 | TAGATAGATG ACAGAGAAAT AGAGAAGACC TAATATTTTT TGTTCATGGC AAATCGCGGG |
| 1381 | TTCGCGGTCG GGTCACACAC GGAGAAGTAA TGAGAAGAGC TGGTAATCTG GGGTAAAAGG |
| 1441 | GTTCAAAAGA AGGTCGCCTG GTAGGGATGG AATACAAGGT TGTCTTGGAG TTTACATTGA |
| 1501 | CCAGATGATT TGGCTTTTTC TCTGTTCAAT TCACATTTTT CAGCGAGAAT CGGATTGACG |
| 1561 | GAGAAATGGC GGGGTGTGGG GTGGATAGAT GGCAGAAATG CTCGCAATGA CCGCGAAAGA |
| 1621 | AAGACTTTAT GGAATAGAAC TACTGGGTGG TGTAAGGATT ACATAGCTAG TCCAATGGAG |
| 1681 | TCCGTTGGAA AGGTAAGAAG AAGCTAAAAC CGGCTAAGTA ACTAGGGAAG AATGATCAGA |
| 1741 | CTTTGATTTC ATGACGTCTG AAAATACTCT GCTGCTTTTT CAGTTGCTTT TTCCCTGCAA |
| 1801 | CCTATCATTT TCCTTTTCAT AAGCCTGCCT TTTCTGTTTT CAGTTATATG AGTTCCGCCG |
| 1861 | AGACTTCCCC AAATTCTCTC CTGGAACATT CTCTATCGCT CTCCTTCCAA GTTGCGCCCC |
| 1921 | CTGGCAGTGC CTAGTAATAT TACCACGCGA CTTATATTCA GTTCCACAAT TTCCAGTGTT |
| 1981 | CGTAGCAAAT ATCATCAGCC ATGGCGAAGG CAGATGGCAG TTTGCTCTAC TATAATCCTC |
| 2041 | ACAATCCACC CAGAAGGTAT TACTTCTACA TGGCTATATT CGCCGTTTCT GTCATTTGCG |
| 2101 | TTTTGTACGG ACCCTCACAA CAATTATCAT CTCCAAAAAT AGACTATGAT CCATTGACGC |
| 2161 | TCCGATCACT TGATTTGAAG ACTTTGGAAG CTCCTTCACA GTTGAGTCCA GGCACCGTAG |
| 2221 | AAGATAATCT TCGaagacaa ttggagtttc attttcctta ccgcagttac gaaccttttc |
| 2281 | cccaacatat ttggcaaacg tggaaagttt ctccctctga tagttccttt ccgaaaaact |
| 2341 | tcaacgactt aggtgaaagt tggctgcaaa ggtccccaaa ttatgatcat tttgtgatac |
| 2401 | ccgatgatgc agcatgggca cttattcacc atgaatacga acgtgtacca gaagtcttgg |
| 2461 | aagctctaga tgctcaccgc aatgctgtta aggttcgtat ggagaaactg ggacttattt |
| 2521 | aattatttag agattttaac ttacatttag attcgataga tccacaggac gggtgtggtc |
| 2581 | gccatgatcg cgtagtcgat agtggctcca agtagcgaag cgggcaggac tgggcggcgg |
| 2641 | ccaaagcggt cggacagtgc tccgagaacg ggtgcgcata gaaattgcat caacgcatat |
| 2701 | agcgctagca gcacgccata gtgactggcg atgctgtcgg aatggacgat atcccgcaag |
| 2761 | aggcccggca gtaccggcat aaccaagcct atgcctacag catccagggt gacggtgccg |
| 2821 | aggatgacga tgagcgcatt gttagatttc atacacggtg cctgactgcg ttagcaattt |
| 2881 | aactgtgata aactaccgca ttaaagctga tcttttttgt agaaatgtct tggtgtcctc |
| 2941 | gtccaatcag gtagccatct ctqaaatatc tqgctccgtt gcaactccga acgacctgct |
| 3001 | ggcaacgtaa aattctccgg ggtaaaactt aaatgtggag taatggaacc agaaacgtct |
| 3061 | cttcccttct ctctccttcc accgcccgtt accgtcccta ggaaatttta ctctgctgga |
| 3121 | gagcttcttc tacggccccc ttgcagcaat gctcttccca gcattacgtt gcgggtaaaa |
| 3181 | cggaggtcgt gtacccgacc tagcagccca gggatggaaa agtcccggcc gtcgctggca |
| 3241 | ataatagcgg gcggacgcat gtcatgagat tattggaaac caccagaatc gaatataaaa |
| 3301 | ggcgaacacc tttcccaatt ttggtttctc ctgacccaaa gactttaaat ttaatttatt |
| 3361 | tgtccctatt tcaatcaatt gaacaactat ttcgcgaaac gatgagattt ccttcaattt |
| 3421 | ttactgctct tttattcgca acatcctccg cattggctgc tccagtcaac actacaacag |
| 3481 | aagatcgaac ggcacaaatt ccggctgaag ctgtcatcgg ttactcagat ttagaagggg |
| 3541 | atttcgatat tgctgttttg ccattttcca acagcacaaa taacgggtta ttgtttataa |
| 3601 | atactactat tgccagcatt gctgctaaag aagaaggggt atctctcgag aaaagagagg |
| 3661 | ctgaagctga attcgccaca aaacgtggat ctcccaaccc tacgagggcg gcagcagtca |
| 3721 | aggccgcatt ccagacgtcg tggaacgctt accaccattt tgcctttccc catgacgacc |
| 3781 | tccacccggt cagcaacagc tttgatgatg agagaaacgg ctggggctcg tcggcaatcg |
| 3841 | atggcttgga cacggctatc ctcatggggg atgccgacat tgtgaacacg atccttcagt |
| 3901 | atgtaccgca gatcaacttc accacgactg cggttgccaa ccaaggatcc tccgtgttcg |
| 3961 | agaccaacat tcggtacctc ggtggcctgc tttctgccta tgacctgttg cgaggtcctt |
| 4021 | tcagctcctt ggcgacaaac cagaccctgg taaacagcct tctgaggcag gctcaaacac |
| 4081 | tggccaaccg cctcaaggtt gcgttcacca ctcccagcgg tgtcccggac cctaccgtct |
| 4141 | tcttcaaccc tactgtccgg agaagtggtg catctagcaa caacgtcgct gaaattggaa |
| 4201 | gcctggtgct cgagtggaca cggttgagcg acctgacggg aaacccgcag tatgcccagc |
| 4261 | ttgcgcagca gggcgagtcg tatctcctga atccaaaggg aagcccggag gcatagcctg |
| 4321 | gcctgattgg aacgtttgtc agcacgagca acggtacctt tcaggatagc agcggcagct |
| 4381 | ggtccggcct catggacagc ttctacgagt acctgatcaa gatgtacctg tacgacccgg |
| 4441 | ttgcgtttgc acactacaag gatcgctggg tccttggtgc cgactcgacc attgggcatc |
| 4501 | tcggctctca cccgtcgacg cgcaaggact tgaccttttt gtcttcgtac aacggacagt |
| 4561 | ctacgtcgcc aaactcagga catttggcca gttttggcgg tggcaacttc atcttgggag |
| 4621 | gcattctcct gaacgagcaa aagtacattg actttggaat caagcttgcc agctcgtact |
| 4681 | ttggcacgta cacccagacg gcttctggaa tcggccccga aggcttcgcg tgggtggaca |
| 4741 | gcgtgacggg cgccggcggc tcgccgccct cgtcccagtc cgggttctac tcgtcggcag |
| 4801 | gattctgggt gacggcaccg tattacatcc tgcggccgga gacgctggag agcttgtact |
| 4861 | acgcataccg cgtcacgggc gactccaagt ggcaggacct ggcgtgggaa gcgttgagtg |
| 4921 | ccattgagga cgcatgccgc gccggcagcg cgtactcgtc catcaacgac gtgacgcagg |
| 4981 | ccaacggcgg gggtgcctct gacgatatgg agagcttctg gtttgccgag gcgctcaagt |
| 5041 | atgcgtacct gatctttgcg gaggagtcgg atgtgcaggt gcaggccacc ggcgggaaca |
| 5101 | aatttgtctt taacacggag gcgcacccct ttagcatccg ttcatcatca cgacggggcg |
| 5161 | gccaccttgc tcacgacgag ttgtaatcta gggcGGCCGC CAGCTTGGGC CCGAACAAAA |
| 5221 | ACTCATCTCA GAACAGGATC TGAATAGCGC CGTCGACCAT CATCATCATC ATCATTGAGT |
| 5281 | TTTAGCCTTA GACATGACTG TTCCTCAGTT CAAGTTGGGC ACTTACGAGA AGACCGGTCT |
| 5341 | TGCTAGATTC TAATCAAGAG GATGTCAGAA TGCCATTTGC CTGAGAGATG CAGGCTTCAT |
| 5401 | TTTTGATACT TTTTTATTTG TAACCTATAT AGTATAGGAT TTTTTTTGTC ATTTTGTTTC |
| 5461 | TTCTCGTACG AGCTTGCTCC TGATCAGCCT ATCTCGCAGC TGATGAATAT CTTGTGGTAG |
| 5521 | GGGTTTGGGA AAATCATTCG AGTTTGATGT TTTTCTTGGT ATTTCCCACT CCTCTTCAGA |
| 5581 | GTACAGAAGA TTAAGTGAGA CCTTCGTTTG TGCGGATGCC CCACACACCA TAGCTTCAAA |
| 5641 | ATGTTTCTAC TCCTTTTTTA CTCTTCGAGA TTTTCTCGGA CTCCGCGCAT CGCCGTACCA |
| 5701 | CTTCAAAACA CCCAAGCACA GCATACTAAA TTTCCCCTCT TTCTTCCTCT AGGGTGTCGT |
| 5761 | TAATTACCCG TACTAAAGGT TTGGAAAAGA AAAAAGAGAC CGCCTCGTTT CTTTTTCTTC |
| 5821 | GTCGAAAAAG GCAATAAAAA TTTTTATCAC GTTTCTTTTT CTTGAAAATT TTTTTTTTTG |
| 5881 | ATTTTTTTCT CTTTCGATGA CCTCCCATTG ATATTTAAGT TAATAAACGG TCTTCAATTT |
| 5941 | CTCAAGTTTC AGTTTCATTT TTCTTGTTCT ATTACAACTT TTTTTACTTC TTGCTCATTA |
| 6001 | GAAAGAAAGC ATAGCAATCT AATCTAAGGG CGGTGTTGAC AATTAATCAT CGGCATAGTA |
| 6061 | TATCGGCATA GTATAATACG ACAAGGTGAG GAACTAAACC ATGGCCAAGC CTTTGTCTCA |
| 6121 | AGAAGAATCC ACCCTCATTG AAAGAGCAAC GGCTACAATC AACAGCATCC CCATCTCTGA |
| 6181 | AGACTACAGC GTCGCCAGCG CAGCTCTCTC TAGCGACGGC CGCATCTTCA CTGGTGTCAA |
| 6241 | TGTATATCAT TTTACTGGGG GACCTTGTGC AGAACTCGTG GTGCTGGGCA CTGCTGCTGC |
| 6301 | TGCGGCAGCT GGCAACCTGA CTTGTATCGT CGCGATCGGA AATGAGAACA GGGGCATCTT |
| 6361 | GAGCCCCTGC GGACGGTGCC GACAGGTGCT TCTCGATCTG CATCCTGGGA TCAAAGCCAT |
| 6421 | AGTGAAGGAC AGTGATGGAC AGCCGACGGC AGTTGGGATT CGTGAATTGC TGCCCTCTGG |
| 6481 | TTATGTGTGG GAGGGCTAAG CACTTCGTGG CCGAGGAGCA GGACTGACAC GTCCGACGCG |
| 6541 | GCCCGACGGG TCCGAGGCCT CGGAGATCCG TCCCCCTTTT CCTTTGTCGA Tatcatgtaa |
| 6601 | ttagttatgt cacgcttaca ttcacgccct ccccccacat ccgctctaac cgaaaaggaa |
| 6661 | ggagttagac aacctgaagt ctaggtccct atttattttt ttatagttat gttagtatta |
| 6721 | agaacgttat ttatatttca aatttttctt ttttttctgt acagacgcgt gtacgcatgt |
| 6781 | accattatac tgaaaacctt gcttgagaag gttttgggac gctcgaaggc tttaatttgc |
| 6841 | aagctggaga ccaacatgtg agcaaaaggc cagcaaaagg ccaggaaccg taaaaaggcc |
| 6901 | gcgttgctgg cgtttttcca taggctccgc ccccctgacg agcatcacaa aaatcgacgc |
| 6961 | tcaagtcaga ggtggcgaaa cccgacagga ctataaagat accaggcgtt tccccctgga |
| 7021 | agctccctcg tgcgctctcc tgttccgacc ctgccgctta ccggatacct gtccgccttt |
| 7081 | ctcccttcgg gaagcgtggc gctttctcaa tgctcacgct gtaggtatct cagttcggtg |
| 7141 | taggtcgttc gctccaagct gggctgtgtg cacgaacccc ccgttcagcc cgaccgctgc |
| 7201 | gccttatccg gtaactatcq tcttgagtcc aacccggtaa gacacgactt atcgccactg |
| 7261 | gcagcagcca ctggtaacag gattagcaga gcgaggtatg taggcggtgc tacagagttc |
| 7321 | ttgaagtggt ggcctaacta cggctacact agaaggacag tatttggtat ctgcgctctg |
| 7381 | ctgaagccag ttaccttcgg aaaaagagtt ggtagctctt gatccggcaa acaaaccacc |
| 7441 | gctggtagcg gtggtttttt tgtttgcaag cagcagatta cgcgcagaaa aaaaggatct |
| 7501 | caagaagatc ctttgatctt ttctacgggg tctgacgctc agtggaacga aaactcacgt |
| 7561 | taagggattt tggtcatgag atcagatcta acatccataa tcgtattcgc cgtttctgtc |
| 7621 | atttgcgttt tgtacggacc ctcacaacaa ttatcatctc caaaaataga ctatgatcca |
| 7681 | ttgacgctcc gatcacttga tttgaagact ttggaagctc cttcacagtt gagtccaggc |
| 7741 | accgtagaag ataatcttCG AAGACAATTG GAGTTTCATT TTCCTTACCG CAGTTACGAA |
| 7801 | CCTTTTCCCC AACATATTTG GCAAACGTGG AAAGTTTCTC CCTCTGATAG TTCCTTTCCG |
| 7861 | AAAAACTTCA AAGACTTAGG TGAAAGTTGG CTGCAAAGGT CCCCAAATTA TGATCATTTT |
| 7921 | GTGATACCCG ATGATGCAGC ATGGGAACTT ATTCACCATG AATACGAACG TGTACCAGAA |
| 7981 | GTCTTGGAAG CTTTCCACCT GCTACCAGAG CCCATTCTAA AGGCCGATTT TTTCAGGTAT |
| 8041 | TTGATTCTTT TTGCCCGTGG AGGACTGTAT GCTGACATGG ACACTATGTT ATTAAAACCA |
| 8101 | ATAGAATCGT GGCTGACTTT CAATGAAACT ATTGGTGGAG TAAAAAACAA TGCTGGGTTG |
| 8161 | GTCATTGGTA TTGAGGCTGA TCCTGATAGA CCTGATTGGC ACGACTGCTA TGCTAGAAGG |
| 8221 | ATACAATTTT GCCAATGGGC AATTCAGTCC AAACGAGGAC ACCCAGCACT GCGTGAACTG |
| 8281 | ATTGTAAGAG TTGTCAGCAC GACTTTACGG AAAGAGAAAA GCGGTTACTT GAACATGGTG |
| 8341 | GAAGGAAAGG ATCGTGGAAG TGATGTGATG GACTGGACGG GTCCAGGAAT ATTTACAGAC |
| 8401 | ACTCTATTTG ATTATATGAC TAATGTCAAT ACAACAGGCC ACTCAGGCCA AGGAATTGGA |
| 8461 | GCTGGCTCAG CGTATTACAA TGCCTTATCG TTGGAAGAAC GTGATGCCCT CTCTGCCCGC |
| 8521 | CCGAACGGAG AGATGTTAAA AGAGAAAGTC CCAGGTAAAT ATGCACAGCA GGTTGTTTTA |
| 8581 | TGGGAACAAT TTACCAACCT GCGCTCCCCC AAATTAATCG ACGATATTCT TATTCTTCCG |
| 8641 | ATCACCAGCT TCAGTCCAGG GATTGGCCAC AGTGGAGCTG GACATTTCAA CCATCACCTT |
| 8701 | GCATATATTA GGCATACATT TGAAGGAAGT TGGAAGGACT AAAGAAAGCT AGAGTAAAAT |
| 8761 | AGATATAGCG AGATTAGAGA ATGAATACCT TCTTCTAAGC GATCGTCCGT CATCATAGAA |
| 8821 | TATCATGGAC TGTATAGTTT TTTTTTTGTA CATATAATGA TTAAACGGTC ATCCAACATC |
| 8881 | TCGTTGACAG ATCTCTCAGT ACGCGAAATC CCTGACTATC AAAGCAAGAA CCGATGAAGA |
| 8941 | AAAAAACAAC AGTAACCCAA ACACCACAAC AAACACTTTA TCTTCTCCCC CCCAACACCA |
| 9001 | ATCATCAAAG AGATGTCGGA ACCAAACACC AAGAAGCAAA AACTAACCCC ATATAAAAAC |
| 9061 | ATCCTGGTAG ATAATGCTGG TAACCCGCTC TCCTTCCATA TTCTGGGCTA CTTCACGAAG |
| 9121 | TCTGACCGGT CTCAGTTGAT CAACATGATC CTCGAAATGG GTGGCAAGAT CGTTCCAGAC |
| 9181 | CTGCCTCCTC TGGTAGATGG AGTGTTGTTT TTGACAGGGG ATTACAAGTC TATTGATGAA |
| 9241 | GATACCCTAA AGCAACTGGG GGACGTTCCA ATATACAGAG ACTCCTTCAT CTACCAGTGT |
| 9301 | TTTGTGCACA AGACATCTCT TCCCATTGAC ACTTTCCGAA TTGACAAGAA CGTCGACTTG |
| 9361 | GCTCAAGATT TGATCAATAG GGCCCTTCAA GAGTCTGTGG ATCATGTCAC TTCTGCCAGC |
| 9421 | ACAGCTGCAG CTGCTGCTGT TGTTGTCGCT ACCAACGGCC TGTCTTCTAA ACCAGACGCT |
| 9481 | CGTACTAGCA AAATACAGTT CACTCCCGAA GAAGATCGTT TTATTCTTGA CTTTGTTAGG |
| 9541 | AGAAATCCTA AACGAAGAAA CACACATCAA CTGTACACTG AGCTCGCTCA GCACATGAAA |
| 9601 | AACCATACGA ATCATTCTAT CCGCCACAGA TTTCGTCGTA ATCTTTCCGC TCAACTTGAT |
| 9661 | TGGGTTTATG ATATCGATCC ATTGACCAAC CAACCTCGAA AAGATGAAAA CGGGAACTAC |
| 9721 | ATCAAGGTAC AAGATCTTCC ACAAGGAATT CGTGGTCATT ATTCTGCCCA AGATGATTAC |
| 9781 | AATTTGTGTT TATCGGTTCA ACCTTTCATT GAATCTGTAG ATGAGACAAC AGGCCAAGAA |
| 9841 | TTTTTCAAAC CTCTGAAAGG TGTATTTGAT GACTTGGAAT CTCGCTTTCC TCACCATACA |
| 9901 | AAGACTTCCT GGAGAGACAG ATTCAGAAAG TTTGCCTCTA AATACGGTGT TCGTCAGTAC |
| 9961 | ATCGCGTATT ATGAAAAGAC TGTTGAACTC AATGGTGTTC CTAATCCGAT GACGAACTTT |
| 10021 | ACCTCAAAGG CTTCCATTGA AAAATTTAGA GAAAGACGCG GGACTTCACG TAACAGTGGC |
| 10081 | CTTCCAGGCC CGGTTGGTGT AGAAGCTGTA AGCTCTTTGG ACCACATATC CCCATTGGTC |
| 10141 | ACATCTAATT CCAATTCTGC AGCTGCTGCA GCTGCTGCCG CAGCAGTTGC AGCCTCTGCC |
| 10201 | TCTGCTTCTT CAGCTCCTAA TACTTCAACT ACCAATTTCT TTGAACAGGA GAATATTGCC |
| 10261 | CAACTTCTCT CTGCACATAA CAACGAGCAG TCTATTGCAG AAGTTATTGA GTCCGCACAG |
| 10321 | AATGTCAACA CCCATGAAAG TGAACCTATA GCTGATCATG TTCGAAAAAA TCTTACAGAC |
| 10381 | GATGAATTGC TTGACAAAAT GGATGATATT TTAAGCTCCA GAAGTCTAGG CGGACTAGAT |
| 10441 | GACTTGATAA AGATCCTCTA CACTGAGCTG GGATTTGCTC ATCGTTATAC CGAATTTCTT |
| 10501 | TTTACCTCAT GTTCTCGTGA TGTGATTTTC TTCCGACCAT TAGTGGAACA TTTCCTTCTT |
| 10561 | ACTGGTGAGT GGGAGCTGGA GAATACTCGT GGCATCTGGA CCGGTCGTCA AGACGAAATG |
| 10621 | CTACGTGCTA GCAATCTAGA TGACCTGCAC AAGTTAATTG ACCTGCATGG GAAAGAACGT |
| 10681 | GTTGAGACCA GAAGAAAAGC CATCAAGGGA GAATGATCAT AAGAAATGAA AAACGTATAA |
| 10741 | GT |

TABLE 5
SEQ ID NO: 54 (top) and SEQ ID NO: 55 (bottom)
AMINO ACID SEQUENCE
MAKADGSLLY YNPHNPPRRY YFYMAIFAVS VICVLYGPSQ
QLSSPKIDYD PLTLRSLDLK TLEAPSQLSP GTVEDNLRRQ
LEFHFPYRSY EPFPQHIWQT WKVSPSDSSF PKNFKDLGES
WLQRSPNYDH FVIPDDAAWE LIHHEYERVP EVLEALDAHR
NAVKVRMEKL GLI
DNA SEQUENCE
ATGGCGAAGG CAGATGGCAG TTTGCTCTAC TATAATCCTC
ACAATCCACC CAGAAGGTAT TACTTCTACA TGGCTATATT
CGCCGTTTCT GTCATTTGCG TTTTGTACGG ACCCTCACAA
CAATTATCAT CTCCAAAAAT AGACTATGAT CCATTGACGC
TCCGATCACT TGATTTGAAG ACTTTGGAAG CTCCTTCACA
GTTGAGTCCA GGCACCGTAG AAGATAATCT TCGAAGACAA
TTGGAGTTTC ATTTTCCTTA CCGCAGTTAC GAACCTTTTC
CCCAACATAT TTGGCAAACG TGGAAAGTTT CTCCCTCTGA
TAGTTCCTTT CCGAAAAACT TCAAAGACTT AGGTGAAAGT
TGGCTGCAAA GGTCCCCAAA TTATGATCAT TTTGTGATAC
CCGATGATGC AGCATGGGAA CTTATTCACC ATGAATACGA
ACGTGTACCA GAAGTCTTGG AAGCTCTAGA TGCTCACCGC
AATGCTGTTA AGGTTCGTAT GGAGAAACTG GGACTTATTT AA

TABLE 6
SEQ ID NO: 56 (top) and SEQ ID NO: 57 (bottom)
AMINO ACID SEQUENCE
MRSDLTSIIV FAVSVICVLY GPSQQLSSPK IDYDPLTLRS LDLKTLEAPS
QLSPGTVEDN LRRQLEFHFP YRSYEPFPQH IWQTWKVSPS DSSFPKNFKD
LGESWLQRSP NYDHFVIPDD AAWELIHHEY ERVPEVLEAF HLLPEPILKA
DFFRYLILFA RGGLYADMDT MLLKPIESWL TFNETIGGVK NNAGLVIGIE
ADPDRPDWHD WYARRIQFCQ WAIQSKRGHP ALRELIVRVV
STTLRKEKSG YLNMVEGKDR GSDVMDWTGP GIFTDTLFDY
MINVNTIGHS GQGIGAGSAY YNALSLEERD ALSARPNGEM LKEKVPGKYA
QQVVLWEQFT NLRSPKLIDD ILILPITSFS PGIGHSGAGD LNHHLAYIRH
TFEGSWKD
DNA SEQUENCE

| | |
|---|---|
| 1 | atgagatcag atctaacatc cataatcgta ttcgccgttt ctgtcatttg cgttttgtac |
| 61 | ggaccatcac aacaattatc atctccaaaa atagactatg atccattgac gctccgatca |
| 121 | cttgatttga agactttgga agctccttca cagttgagtc caggcaccgt agaagataat |
| 181 | CTTCGAAGAC AATTGGAGTT TCATTTTCCT TACCGCAGTT ACGAACCTTT TCCCCAACAT |
| 241 | ATTTGGCAAA CGTGGAAAGT TTCTCCCTCT GATAGTTCCT TTCCGAAAAA CTTCAAAGAC |
| 301 | TTAGGTGAAA GTTGGCTGCA AAGGTCCCCA AATTATGATC ATTTTGTGAT ACCCGATGAT |
| 361 | GCAGCATGGG AACTTATTCA CCATGAATAC GAACGTGTAC CAGAAGTCTT GGAAGCTTTC |
| 421 | CACCTGCTAC CAGAGCCCAT TCTAAAGGCC GATTTTTTCA GGTATTTGAT TCTTTTTGCC |
| 481 | CGTGGAGGAC TGTATGCTGA CATGGACACT ATGTTATTAA AACCAATAGA ATCGTGGCTG |
| 541 | ACTTTCAATG AAACTATTGG TGGAGTAAAA AACAATGCTG GGTTGGTCAT TGGTATTGAG |
| 601 | GCTGATCCTG ATAGACCTGA TTGGCACGAC TGGTATGCTA GAAGGATACA ATTTTGCCAA |
| 661 | TGGGCAATTC AGTCCAAACG AGGACACCCA GCACTGCGTG AACTGATTGT AAGAGTTGTC |
| 721 | AGCACGACTT TACGGAAAGA GAAAAGCGGT TACTTGAACA TGGTGGAAGG AAAGGATCGT |
| 781 | GGAAGTGATG TGATGGACTG GACGGGTCCA GGAATATTTA CAGACACTCT ATTTGATTAT |
| 841 | ATGACTAATG TCAATACAAC AGGCCACTCA GGCCAAGGAA TTGGAGCTGG CTCAGCGTAT |
| 901 | TACAATGCCT TATCGTTGGA AGAACGTGAT GCCCTCTCTG CCCGCCCGAA CGGAGAGATG |
| 961 | TTAAAAGAGA AAGTCCCAGG TAAATATGCA CAGCAGGTTG TTTTATGGGA ACAATTTACC |
| 1021 | AACCTGCGCT CCCCCAAATT AATCGACGAT ATTCTTATTC TTCCGATCAC CAGCTTCAGT |
| 1081 | CCAGGGATTG GCCACAGTGG AGCTGGAGAT TTGAACCATC ACCTTGCATA TATTAGGCAT |
| 1141 | ACATTTGAAG GAAGTTGGAA GGACTAA |

What is claimed is:

1. A purified preparation of a protein made by a method which comprises:
a. expressing the protein from an engineered stable strain of Pichia pastoris in a cell culture, wherein the strain comprises a mutant OCH1 allele comprising a nucleotide sequence encoding a mutant OCH1 protein, wherein said mutant OCH1 protein (i) comprises a catalytic domain comprising amino acid residues 45-404 of the wild type OCH1 protein of the amino acid sequence of SEQ ID NO: 2, or (ii) comprises a catalytic domain comprising at least 90% amino acid sequence identity to the amino acid residues 45-404 of the wild type OCH1 protein of the amino acid sequence of SEQ ID NO: 2, and wherein said mutant OCH1 protein has α-1, 6-mannosyltransferase activity, and wherein said mutant OCH1 protein lacks an N-terminal sequence for targeting the mutant OCH1 protein to the Golgi apparatus; and
b. isolate the protein from the cell culture to obtain the purified preparation of the protein, wherein the purified preparation is substantially homogeneous in the N-glycan structures, with Man5GlcNac2 constituting at least 80% of the N-glycan structures.

2. The purified preparation of a protein of claim 1, wherein the protein is selected from the group consisting of trastuzumab, Candida antarctica lipase A, Candida antarctica lipase B, and human serum transferrin.

3. The purified preparation of a protein of claim 1, wherein Man5GlcNac2 constitutes at least 85% of the N-glycan structures in the purified preparation of the protein.

4. The purified preparation of a protein of claim 1, wherein Man5GlcNac2 constitutes at least 90% of the N-glycan structures in the purified preparation of the protein.

5. The purified preparation of a protein of claim 1, wherein the catalytic domain comprises at least 95% amino acid sequence identity to the amino acid residues 45-404 of the amino acid sequence of SEQ ID NO: 2.

6. The purified preparation of a protein of claim 1, wherein the mutant OCH1 protein lacks a membrane anchor domain at the N-terminal region.

7. The purified preparation of a protein of claim 1, wherein said mutant OCH1 protein comprises the amino acid sequence of SEQ ID NO: 3.

8. The purified preparation of a protein of claim 1, wherein said engineered stable strain further comprises a nucleic acid encoding for and expressing an α-1,2-mannosidase.

9. The purified preparation of a protein of claim 8, wherein said nucleic acid coding for and expressing said α-1,2-mannosidase is integrated at the OCH1 locus of the engineered stable strain.

10. The purified preparation of a protein of claim 9, wherein the OCH1 locus comprises the nucleotide sequence of SEQ ID NO: 1.

11. A purified preparation of a protein, wherein the protein is selected from the group consisting of trastuzumab, Candida antarctica lipase A, Candida antarctica lipase B, and human serum transferrin, and wherein the purified preparation is substantially homogeneous in the N-glycan structures, with Man5GlcNac2 constituting at least 80% of the N-glycan structures.

12. The purified preparation of a protein of claim 11, wherein Man5GlcNac2 constitutes at least 85% of the N-glycan structures in the purified preparation of the protein.

13. The purified preparation of a protein of claim 11, wherein Man5GlcNac2 constitutes at least 90% of the N-glycan structures in the purified preparation of the protein.

14. A method for obtaining a purified preparation of a protein, comprising:
a. expressing the protein from an engineered stable strain of Pichia pastoris in a cell culture, wherein the strain comprises a mutant OCH1 allele comprising a nucleotide sequence encoding a mutant OCH1 protein, wherein said mutant OCH1 protein (i) comprises a catalytic domain comprising amino acid residues 45-404 of the wild type OCH1 protein of the amino acid sequence of SEQ ID NO: 2, or (ii) comprises a catalytic domain comprising at least 90% amino acid sequence identity to the amino acid residues 45-404 of the wild type OCH1 protein of the amino acid sequence of SEQ ID NO: 2, and wherein said mutant OCH1 protein has α-1, 6-mannosyltransferase activity, and wherein said mutant OCH1 protein lacks an N-terminal sequence for targeting the mutant OCH1 protein to the Golgi apparatus; and
b. isolating the protein from the cell culture to obtain the purified preparation of the protein, wherein the purified preparation is substantially homogeneous in the N-glycan structures, with Man5GlcNac2 constituting at least 80% of the N-glycan structures.

15. The method of claim 14, wherein the protein is selected from the group consisting of trastuzumab, Candida antarctica lipase A, Candida antarctica lipase B, and human serum transferrin.

16. The method of claim 14, wherein Man5GlcNac2 constitutes at least 85% of the N-glycan structures in the purified preparation of the protein.

17. The method of claim 14, wherein Man5GlcNac2 constitutes at least 90% of the N-glycan structures in the purified preparation of the protein.

18. The method of claim 14, wherein the mutant OCH1 protein lacks a membrane anchor domain at the N-terminal region.

19. The method of claim 14, wherein said mutant OCH1 protein comprises the amino acid sequence of SEQ ID NO: 3.

20. The method of claim 14, wherein said engineered stable strain further comprises a nucleic acid encoding for and expressing an α-1,2-mannosidase.
